(19) United States
(12) Patent Application Publication
(10) Pub. No.: US 2006/0088830 A1
(43) Pub. Date: Apr. 27, 2006

(54) Protein biomarkers that distinguish prostate cancer from non-malignant cells

(75) Inventors: George L Wright JR., Virginia Beach, VA (US); Lisa H Cazares, Chesapeake, VA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignee: Eastern Virginia Medical School, NorfolK, VA (US)

(21) Appl. No.: 10/505,367

(22) PCT Filed: Feb. 20, 2003
(86) PCT No.: PCT/US03/05042

§ 371 Date: Sep. 16, 2005

Related U.S. Application Data
(60) Provisional application No. 60/358,986, filed on Feb. 21, 2002.

(51) Int. Cl. C12Q 1/68; G01N 33/574; G06F 19/00; G01N 33/00

(52) U.S. Cl. 435/6; 435/7.23; 436/86; 702/19

(57) ABSTRACT

This invention provides organic biomolecule markers (e.g., proteins) useful for differentiating prostate cancer, prostate intraepithelial neoplasia or benign prostate hyperplasia, from a negative diagnosis (i.e. normal and benign prostate epithelial cells).

### CROSS-REFERENCES TO RELATED APPLICATIONS

This application claims the benefit of U.S. Provisional Application No.60/358,986 filed Feb. 21, 2002, which is incorporated herein by reference.

### STATEMENT AS TO RIGHTS TO INVENTIONS MADE UNDER FEDERALLY SPONSORED RESEARCH AND DEVELOPMENT

This invention was made with government support under grant no. UO1 CA85067 awarded by the National Cancer Institute. The government may have certain rights in this invention.

### BACKGROUND OF THE INVENTION

Prostate cancer is the most common form of cancer in males. It typically afflicts aging males, but it can afflict males of all ages. A significant number of males die from prostate cancer every year, and it is the second leading cause of cancer deaths in men. Early diagnosis of prostate cancer in patients reduces the likelihood of death.

The effectiveness of any diagnostic test depends upon its specificity and selectivity. That is, what is the relative ratio of true positive diagnoses, true negative diagnoses, false positive diagnoses and false negative diagnoses? Methods of increasing the percentage of true positive and true negative diagnoses for any condition are desirable medical goals. In the case of prostate cancer, the present diagnostic tests are not completely satisfactory, in that they provide significant numbers of false positive and false negative results.

Conventionally, prostate cancer is diagnosed using prostate specific antigen (PSA) as a marker. In general, PSA levels above 4 ng/ml are suggestive of prostate cancer while levels above 10 ng/ml are highly suggestive of prostate cancer. However, if the cancer is in its early stages, some prostate cancer patients exhibit normal PSA levels at the time of diagnosis. Since conventional PSA tests detect abnormal levels of PSA, conventional PSA tests may not be able to detect the presence of prostate cancer if it is in its early stages. This results in a false negative diagnosis. The inability of conventional PSA tests to diagnose the presence of prostate cancer in some instances (e.g., in the early stages of the disease) can be detrimental to the patient. Moreover, many individuals with elevated levels of PSA in the blood serum may not have prostate cancer, but may instead have benign prostate hyperplasia (BPH) (i.e., a benign tumor) or prostate intraepithelial neoplasia (PIN). This results in a false positive diagnosis. In order to determine if a person has prostate cancer, rather than BPH or PIN, additional immunoassays using other antibodies and/or biopsies of the prostate tissue are performed. These additional tests are time consuming and expensive for both patients and their care providers.

There is some consensus in the medical community that better diagnosis will result from the discovery of more disease markers that can be used alone or in combination to increase the specificity and selectivity of diagnostic tests.

### SUMMARY OF THE INVENTION

Several organic biomolecules (e.g., proteins, glycoproteins, nucleoproteins and the like) have been discovered that possess molecular weights capable of functioning as markers in prostate cancer (“PCA”), prostate intraepithelial neoplasia (“PIN”), or benign prostate hyperplasia (“BPH”) versus a negative diagnosis. Compared to a negative diagnosis, the markers are, variously, more frequently detected, less frequently detected, or differentially detected. The detection and amount (either absolute or relative) of these markers, alone or in combination, in patient samples provides information that the diagnostician can correlate with probable diagnoses of prostate cancer, benign prostate hyperplasia or with a negative diagnosis (e.g., normal or disease-free). Each marker can be distinguished from other biomolecular masses as the source from which the markers are derived is controlled and/or particular purification procedures are employed that minimize the potential of false positives, as described herein. As is also described herein, there are several methods that allow resolution and detection of the marker molecular weights. In preferred embodiments, the method of resolution involves Surface-Enhanced Laser Desorption/Ionization mass spectrometry, in which the surface of the mass spectrometry probe plays an active role in the desorption and ionization of the analyte.

A set of markers was identified in prostate epithelial cell lysates derived from laser capture microdissection (LCM). The Marker Set includes the following: Marker EP1: 3448±19 Da, Marker EP2: 4036±22 Da, Marker EP3: 4361±24 Da, Marker EP4: 4413±24 Da, Marker EP5: 4639±26 Da, Marker EP6: 4749 i 26 Da, Marker EP7: 4827±27 Da, Marker EP8: 5666±31 Da, Marker EP9: 8445±46 Da, Marker EP10: 11744±65 Da, Marker EP11: 14696±81 Da, Marker EP12: 24184±133 Da, Marker EP13: 48308±266 Da, and Marker EP14: 53830±296 Da. These markers are further characterized by the ability to bind to a metal chelate adsorbent and washed with an eluant at neutral pH. This implies that they contain metal binding residues. Markers EP2, EP3, EP5, EP6 and EP12 occur in a greater frequency and abundance in the PIN and PCA cell lysates. Markers EP4, EP8, EP13, and EP14 appear in greater abundance in normal and BPH cell lysates. Marker EP10 had a decreased abundance in BPH, PIN, and PCA cell lysates. Markers EP5 and EP12 are found only in PCA cell lysates.

The in addition to mass characteristics, the markers may also be characterized by affinity characteristics. For example, the molecular weight and metal binding characteristics are provided herein. We point out that molecular weight and metal binding are characteristic properties of these markers and not limitations on means of detection or isolation. Furthermore, once partial amino acid sequences are obtained, each marker can be identified unambiguously by matching the sequence in a protein database.

One aspect of the invention is a method of qualifying a prostate cancer status in a subject comprising: a) measuring at least one marker in a sample from the subject, wherein the marker is selected from the group consisting of those markers noted above as being identified in prostate epithelial cell lysates and combinations thereof; and, b) correlating the measurement with prostate cancer status.

In one embodiment of this method, the prostate cancer status is selected from the group consisting of prostate cancer (PCA), prostate intraepithelial neoplasia (PIN), and benign prostate hyperplasia (BPH). In another embodiment the sample is prostate tissue extract. Still another embodiment is where measuring comprises determining the mass of the protein, which an be by mass spectrometry, preferably is gas phase ion spectrometry, more preferably laser desorption ionization mass spectrometry.

Samples used in the method may be selected from the group consisting of blood, serum, urine, prostatic fluid, seminal fluid, semen, and prostate tissue.

Measuring marker amount may further comprise i) fractionating the sample; ii) binding a fraction of the sample to an adsorbent; and, iii) comprises detecting the protein marker by gas phase ion spectrometry. Alternatively, marker amount may be detected by immunoassay.

The absorbent used in the methods may be selected from the group consisting of a hydrophilic adsorbent, a metal chelate adsorbent, and a strong anion exchange adsorbent.

In another embodiment of the method, measuring the biomarker(s) comprises i) embedding a portion of a tissue specimen harvested from a patient in OCT and freezing the specimen; ii) obtaining cryosections from the tissue specimen; iii) obtaining cell samples from the cryosections by laser capture microdissection; iv) mixing cell samples from step (d) with lysis buffer thereby producing cell lysates; v) diluting and vortexing the cell lysates; vi) centrifuging the vortexed cell lysates thereby producing a supernatant fraction; vii) binding the supernatant fraction to an adsorbent; and, viii) comprising detecting the protein marker using gas phase ion spectrometry. The adsorbent used may be selected from the group consisting of a hydrophilic adsorbent, a metal chelate adsorbent, and a strong anion exchange adsorbent. The sample may comprise any number of marker combinations, for example simply marker EP8, markers EP2 and EP3, EP2 and EP5, markers EP3 and EP5, markers EP2 and EP6, markers EP3 and EP6, markers EP5 and EP6, markers EP2, EP3 and EP5, markers EP2, EP3 and EP6, or markers EP2, EP3, EP4, EP5, EP6, and EP8.

In one embodiment, correlating the measurement with prostate cancer status includes: i) generating data for each protein marker with the mass spectrometer, the data comprising a mass/charge ratio and an amount determination for each ion corresponding to each protein marker; ii) transforming the data into computer-readable form; and, iii) executing an algorithm with a programmable digital computer, wherein the algorithm determines closeness-of-fit between the computer-readable data and a data set indicating a diagnosis of PCA, PIN, BPH or a negative diagnosis.

In another aspect this invention provides a method for aiding in a diagnosis of prostate cancer, prostate intraepithelial neoplasia, or benign prostate hyperplasia. The method involves: a) detecting at least one protein marker selected from the Marker Set (as defined herein) in a sample from a subject; and b) correlating the detection of the marker or markers with a probable diagnosis of prostate cancer, prostate intraepithelial neoplasia, benign prostate hyperplasia or a negative diagnosis, wherein the correlation takes into account the relative detectability of the marker or markers in each diagnosis.

In certain embodiments, the sample may be selected from seminal plasma, blood, serum, urine, prostatic fluid, seminal fluid, semen, and prostate tissue. In the preferred embodiment, the marker or markers is detected in cell lysates from prostate tissue. In other embodiments, the marker or markers are detected by gas phase ion spectrometry or, more particularly, laser desorption mass spectrometry. In another embodiment, the marker or markers are detected by immunoassay. In other embodiments, the method comprises detecting a plurality of the markers. In other embodiments, the method involves detecting at least marker EP2 and a second marker selected from EP3, EP5, or EP6. In another embodiment, the method comprises detecting at least marker EP3 and a second marker selected from EP5 or EP6. In another embodiment, the method comprises detecting at least marker EP5 and EP6. In another embodiment, the method comprises detecting at least markers EP2, EP3 and EP5. In another embodiment, the method comprises detecting at least markers EP2, EP3 and EP6. In another embodiment the method comprises: i) generating data on the sample with the mass spectrometer indicating intensity of signal for mass/charge ratio, ii) transforming the data into computer-readable form; and iii) executing an algorithm with a programmable digital computer, wherein the algorithm detects signal in the computer-readable data and wherein detecting the markers is indicative of a diagnosis of PCA, PIN, BPH or a negative diagnosis

In another aspect this invention provides a method for detecting at least one protein marker from the Marker Set in a sample, wherein the method comprises detecting the marker or markers by gas phase ion spectrometry.

In one embodiment the method comprises: i) generating data on the sample with a mass spectrometer indicating intensity of signal for mass/charge ratio, ii) transforming the data into computer-readable form; and iii) executing an algorithm with a programmable digital computer wherein the algorithm detects signal in the computer-readable data representing the marker or markers and wherein detecting the markers is indicative of a diagnosis of PCA, PIN, BPH or a negative diagnosis.

In another aspect of this invention, a marker or multiple markers are detected in samples and an artificial intelligence program such as fuzzy logic, cluster analysis, or neural network are used to analyze the data.

In another embodiment the method further comprises i) before detecting the marker or markers, fractionating a sample comprising the marker or markers by contacting the sample with a substrate comprising an adsorbent that retains the marker or markers and removing unretained sample; and ii) desorbing and ionizing the retained markers from the adsorbent during mass spectrometry. In certain embodiments of this method the substrate is a mass spectrometer probe comprising the adsorbent on a probe surface. In another embodiment, the substrate is a resin, and, after fractionating the sample, the resin with the marker or markers retained by the adsorbent is placed on a mass spectrometer probe for desorption and ionization by the mass spectrometer. In another embodiment the adsorbent is selected from a hydrophilic adsorbent, e.g., an anionic adsorbent, and a metal chelate adsorbent, e.g., a copper chelate adsorbent.

In one embodiment involving laser desorption/ionization mass spectrometry the method comprises: i) providing a probe adapted for use with a mass spectrometer comprising an adsorbent attached thereto; ii) contacting the marker or markers with the adsorbent; iii) desorbing and ionizing the marker or markers from the probe; and, iv) detecting the desorbed/ionized marker or markers with the mass spectrometer. In another embodiment the method comprises: i) providing a substrate comprising an adsorbent attached thereto; ii) contacting the marker or markers with the adsorbent; iii) placing the substrate on a probe adapted for use with a mass spectrometer comprising an adsorbent attached thereto; iv) desorbing and ionizing the marker or markers from the probe; and, v) detecting the desorbed/ionized marker or markers with the mass spectrometer. In other embodiments the adsorbent is a hydrophilic adsorbent (e.g., silicon oxide) or a metal chelate adsorbent (e.g., copper chelate). In another embodiment, the adsorbent comprises an antibody that specifically binds to the marker.

In another aspect the invention provides a purified protein selected from the protein markers of the Marker Set. In one embodiment, the purified protein is produced by a process comprising fractionating a sample comprising the marker or markers by size exclusion chromatography and/or anion exchange chromatography, then collecting a fraction that includes the marker or markers.

In another aspect this invention provides a kit comprising: (1) an adsorbent attached to a substrate, wherein the adsorbent retains a protein marker selected from the Marker Set; and (2) instructions to detect the marker or markers by contacting a sample with the adsorbent and detecting the marker or markers retained by the adsorbent. In one embodiment of this invention the substrate is a probe for a gas phase ion spectrometer having a surface on which the adsorbent is attached. The adsorbent can be, e.g., a metal chelating adsorbent (e.g., IMAC3 CuII chip). In another embodiment the kit further comprises (1) an eluant wherein the marker or markers are retained on the adsorbent when washed with the eluent, or (2) instructions for washing the adsorbent with the eluent after contacting the adsorbent with the marker or markers.

### BRIEF DESCRIPTION OF THE DRAWINGS

FIG. 1: Representative SELDI spectra of one LCM sample from PCA epithelial cells in m/z range of (a) 3000-10,000, (b) 10,000 to 20,000 and (c) 20,000 to 80,000. An average of 70 peaks were detected for each cell lysate analyzed.

FIG. 2a: Representative spectra and gel-view of matched prostate epithelial cell lysates showing protein alterations in the mass/charge(m/z) range of 4000-6000. (arrows indicate over-expressed peaks in PIN and PCA.)

FIG. 2b: SELDI gelview protein profiles of lysates prepared from cells procured from different prostatectomy specimens. The red box identifies a peak with an average mass of 4749 Da that appears to be more abundant in PIN and PCA epithelial cells. Replicate samples(*) show good reproducibility of the protein patterns. The different protein patterns observed for two different PCA foci in Patient #2 and the two PIN foci in Patient #3, may be the result of genetic heterogeneity

FIG. 2c: Representative spectra and gel-views of matched prostate epithelial cell lysates showing increased abundance of a peak at m/z of 5666 in the BPH sample.

FIG. 3: Example of protein peaks found between 20,000-70,000 Da Differential expression of a peak at 28400 Da, thought to be PSA was observed in epithelial cell lysates, but absent in lysates from adjacent stromal cells. The intracellular free PSA appears to be under-expressed in the PIN and PCA epithelial cell lysates. The serum albumin peak at 66800 Da was present in each sample.

FIG. 4: Expression levels of peptide/proteins compared to the expression levels in matched normal epithelial cells. The average expression in normal cell lysates was subtracted from the expression levels found in the other cell types. Upward bars indicate over-expression, downward bars indicate under-expression.

### DESCRIPTION OF THE SPECIFIC EMBODIMENTS

I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Marker” in the context of the present invention refers to an organic biomolecule, particularly a polypeptide, which is differentially present in a sample taken from patients having prostate cancer, prostate intraepithelial neoplasia, or benign prostate hyperplasia as compared to a comparable sample taken from subjects who do not have prostate cancer (e.g., negative diagnosis, normal or healthy subject). For example, a marker can be a polypeptide (having a particular apparent molecular weight) which is present at an elevated or decreased level in samples of prostate cancer patients compared to samples of patients with a negative diagnosis.

“Organic biomolecule” refers to an organic molecule of biological origin, e.g., steroids, amino acids, nucleotides, sugars, polypeptides, polynucleotides, complex carbohydrates or lipids.

The phrase “differentially present” refers to differences in the quantity of a polypeptide (of a particular apparent molecular weight) present in a sample taken from patients having prostate cancer as compared to a comparable sample taken from patients who do not have prostate cancer (e.g., have benign prostate hyperplasia). A polypeptide is differentially present between the two samples if the amount of the polypeptide in one sample is significantly different from the amount of the polypeptide in the other sample. For example, a polypeptide is differentially present between the two samples if it is present in an amount (e.g., concentration, mass, molar amount, etc.) at least about 150%, at least about 200%, at least about 500% or at least about 1000% greater than it is present in the other sample, or if it is detectable in one sample and not detectable in the other. Any polypeptides that are differentially present in samples taken from prostate cancer patients as compared to subjects who do not have prostate cancer (e.g., benign prostate hyperplasia patients) can be used as markers.

“Diagnostic” means identifying the presence or nature of a pathologic condition. Diagnostic methods differ in their sensitivity and specificity. “Sensitivity” is defined as the percent of diseased (Benign Prostate Hyperplasia (BPH)/Prostate Intraepithelial Neoplasia (PIN)/Prostate Adenocarcinoma (PCA)) cell types for which the biomarker of interest is present, (true positive/total number of diseased lysates×100). “Specificity” is defined as the percent of cell types for which the biomarker of interest is not positive, (true negative/total number of lysates without disease×100). Diseased individuals not detected by the assay are “false negatives.” Subjects who are not diseased and who test negative in the assay, are termed “true negatives.” While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of prostate cancer. A diagnostic amount can be either in absolute amount (e.g., μg/ml) or a relative amount (e.g. relative intensity of signals).

A “control amount” of a marker can be any amount or a range of amount which is to be compared against a test amount of a marker. For example, a control amount of a marker can be the amount of a marker (e.g., seminal basic protein) in a prostate cancer patient, a BPH patient or a person without prostate cancer or BPH. A control amount can be either in absolute amount (e.g., μg/ml) or a relative amount (e.g., relative intensity of signals).

“Probe” refers to a device that, when positionally engaged in an interrogatable relationship to an ionization source, e.g., a laser desorption/ionization source, and in concurrent communication at atmospheric or sub-atmospheric pressure with a detector of a gas phase ion spectrometer, can be used to introduce ions derived from an analyte into the spectrometer. As used herein, the “probe” is typically reversibly engageable (e.g., removably insertable) with a probe interface that positions the probe in an interrogatable relationship with the ionization source and in communication with the detector. A probe will generally comprise a substrate comprising a sample presenting surface on which an analyte is presented to the ionization source.

“Substrate” or “probe substrate” refers to a solid phase onto which an adsorbent can be provided (e.g., by attachment, deposition, etc.)

“Ionization source” refers to a device that directs ionizing energy to a sample presenting surface of a probe to desorb and ionize analytes from the probe surface into the gas phase. The preferred ionization source is a laser (used in laser desorption/ionization), in particular, nitrogen lasers, Nd-Yag lasers and other pulsed laser sources. Other ionization sources include fast atoms (used in fast atom bombardment), plasma energy (used in plasma desorption) and primary ions generating secondary ions (used in secondary ion mass spectrometry).

“Gas phase ion spectrometer” refers to an apparatus that detects gas phase ions. In the context of this invention, gas phase ion spectrometers include an ionization source used to generate the gas phase ions. Gas phase ion spectrometers include, for example, mass spectrometers, ion mobility spectrometers, and total ion current measuring devices.

“Gas phase ion spectrometry” refers to a method comprising employing an ionization source to generate gas phase ions from an analyte presented on a sample presenting surface of a probe and detecting the gas phase ions with a gas phase ion spectrometer.

“Mass spectrometer” refers to a gas phase ion spectrometer that measures a parameter which can be translated into mass-to-charge ratios of gas phase ions. Mass spectrometers generally include an inlet system, an ionization source, an ion optic assembly, a mass analyzer, and a detector. Examples of mass spectrometers are time-of-flight, magnetic sector, quadrapole filter, ion trap, ion cyclotron resonance, electrostatic sector analyzer and hybrids of these.

“Mass spectrometry” refers to a method comprising employing an ionization source to generate gas phase ions from an analyte presented on a sample presenting surface of a probe and detecting the gas phase ions with a mass spectrometer.

“Laser desorption mass spectrometer” refers to a mass spectrometer which uses laser as a means to desorb, volatilize and ionize an analyte.

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions also are referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of a marker from all other markers in a mixture. Rather, any separation that allows the distinction between at least two markers suffices.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavidin. The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., P. D. Fahrlander and A. Klausner, Bio/Technology 6:1165 (1988)). Quantitation of the signal is achieved by, e.g., scintillation counting, densitometry, or flow cytometry.

“Measure” in all of its grammatical forms, refers to detecting, quantifying or qualifying the amount (including molar amount), concentration or mass of a physical entity or chemical composition either in absolute terms in the case of quantifying, or in terms relative to a comparable physical entity or chemical composition.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

II. Gas Phase Ion Spectrometry

In one aspect, the invention provides methods for detecting markers which are differentially present in samples from patients with prostate cancer, prostate intraepithelial neoplasia, benign prostate hyperplasia and with negative diagnoses. Any one or combination of markers described are within the scope of this aspect of this invention and can be detected. The methods for detecting these markers have many applications. For example, one marker or combination of markers can be measured to differentiate between PCA or PIN and BPH, and thus are useful as an aid in the diagnosis of prostate cancer in a patient. In another example, the present methods for detecting these markers can be applied to in vitro prostate cancer cells or in vivo animal models for prostate cancer to assay for and identify compounds that modulate expression of these markers. In one embodiment of the detection method, the marker or markers are detected by gas phase ion spectrometry. In a preferred embodiment, the marker or markers are detected by mass spectrometry and, in particular, laser desorption mass spectrometry.

Probes suitable for use in the invention are described in, e.g., U.S. Pat. No. 5,617,060 (Hutchens and Yip) and U.S. Pat. No. 6,225,047 (Hutchens and Yip). In one embodiment, a substrate comprising an adsorbent can be in the form of a probe, which is removably insertable into a gas phase ion spectrometer. For example, a substrate can be in the form of a strip with adsorbents on its surface. In another embodiment, a substrate comprising an adsorbent can be positioned onto another substrate to form a probe, which is removably insertable into a gas phase ion spectrometer. For example, a substrate comprising an adsorbent can be a solid phase, such as a polymeric or glass bead with a functional group for binding a marker, which can be subsequently positioned on a second substrate to form a probe. For example, the second substrate can be in the form of a strip, or a plate having a series of wells at a predetermined addressable locations. One advantage of this embodiment is that the marker can be adsorbed to the first substrate in one physical context, and transferred to the second substrate, which can then be submitted for analysis by gas phase ion spectrometry. The probe can be in any shape as long as it is removably insertable into a gas phase ion spectrometer.

The probe can also be adapted for use with inlet systems and detectors of a gas phase ion spectrometer. For example, the probe can be adapted for mounting in a horizontally, vertically and/or rotationally translatable carriage that moves the probe to a successive position without requiring repositioning of the probe by hand.

The probe substrate is preferably made of a material that is capable of supporting adsorbents. For example, the probe substrate material can include, but is not limited to, insulating materials (e.g., glass, ceramic), semi-insulating materials (e.g., silicon wafers), or electrically conducting materials (e.g., metals, such as copper, brass, steel, aluminum, gold, or electrically conductive polymers), organic polymers, biopolymers, or any combinations thereof.

The probe substrate surface can be conditioned to bind markers. For example, in one embodiment, the surface of the probe substrate can be conditioned (e.g., chemically or mechanically such as roughening) to place adsorbents on the surface. The adsorbent comprises functional groups for binding with a marker. In some embodiments, the substrate material itself can also contribute to adsorbent properties and may be considered part of an “adsorbent.”

Any number of different adsorbents can be used as long as they have binding characteristics suitable for binding the markers of the present invention. The adsorbents can comprise a hydrophobic group, a hydrophilic group, a cationic group, an anionic group, a metal ion chelating group, or antibodies which specifically bind to antigens, or a combination thereof (sometimes referred to as “a mixed mode” adsorbent). Exemplary adsorbents comprising a hydrophobic group include matrices having aliphatic hydrocarbons, e.g., C₁-C₁₈ aliphatic hydrocarbons and matrices having aromatic hydrocarbon functional group such as phenyl groups. Exemplary adsorbents comprising a hydrophilic group include silicon oxide (i.e., glass), or hydrophilic polymers such as polyethylene glycol, dextran, agarose, or cellulose. Exemplary adsorbents comprising a cationic group include matrices of secondary, tertiary or quaternary amines. Exemplary adsorbents comprising an anionic group include matrices of sulfate anions (SO₃⁻) and matrices of carboxylate anions (i.e., COO⁻) or phosphate anions (OPO₃⁻). Exemplary adsorbents comprising metal chelating groups include organic molecules that have one or more electron donor groups which form coordinate covalent bonds with metal ions, such as copper, nickel, cobalt, zinc, iron, and other metal ions such as aluminum and calcium. Exemplary adsorbents comprising an antibody include antibodies that are specific for any one of the markers provided herein. In preferred embodiments, adsorbents are substantially similar to or the same as the adsorbents which were used to enrich and identify the markers.

One useful chip is the Normal Phase ProteinChip® array, available from Ciphergen Biosystems, Inc. (Palo Alto, Calif.). The normal phase chip has a hydrophilic adsorbent comprising silicon oxide (SiO₂) on the substrate surface. Silicon oxide can be applied to the surface by any of a number of well known methods. These methods include, for example, vapor deposition, e.g., sputter coating. A preferred thickness for such a probe is about 9000 Angstroms.

Another useful chip is the SAX1 ProteinChip® array made by Ciphergen Biosystems, Inc. in Palo Alto, Calif. The SAX1 protein chips are fabricated from SiO₂ coated aluminum substrates. In the process, a suspension of quaternary ammonium polystryenemicrospheres in distilled water is deposited onto the surface of the chip (1 mL/spot, two-times). After air drying (room temperature, 5 minutes), the chip is rinsed with deionized water and air dried again (room temperature, 5 minutes).

Another useful chip is the IMAC3 (Immobilized Metal Affinity Capture, nitrilotriacetic acid on surface) ProteinChip® array, also available from Ciphergen Biosystems, Inc. The chips are produced as follows: 5-Methacylamido-2-(N,N-biscarboxymethaylamino)pentanoic acid (7.5 wt %),Acryloyltri-(hydroxymethyl)methylamine (7.5 wt %) and N,N′-methylenebisacrylamide (0.4 wt %) are photo-polymerized using-(−)riboflavin (0.02 wt %) as a photo-initiator. The monomer solution is deposited onto a rough etched, glass coated substrate (0.4 mL, twice) and irradiated for 5 minutes with a near UV exposure system (Hg short arc lamp, 20 mW/cm² at 365 nm). The surface is washed with a solution of sodium chloride (1 M) and then washed twice with deionized water.

The IMAC3 with Cu(II) is activated as follows. The surface is treated with a solution of CuSO₄ (100 mM, 10 μL/spot) and mixed on a high frequency mixer for 10 minutes. After removing the CuSO₄ solution, the chips are rinsed with water and each spot is treated with a solution of sodium acetate (100 mM, pH 4.0, 10 μl per spot). The sodium acetate is removed and the surface is washed with a stream of deionized water (15 sec/chip).

Adsorbents can be placed on the probe substrate in continuous or discontinuous patterns. If continuous, one or more adsorbents can be placed on the substrate surface. If multiple types of adsorbents are used, the substrate surface can be coated such that one or more binding characteristics vary in one or two-dimensional gradient. If discontinuous, plural adsorbents can be placed in predetermined addressable locations on the substrate surface. The addressable locations can be arranged in any pattern, but are preferably in regular pattern, such as lines, orthogonal arrays, or regular curves (e.g., circles). Each addressable location may comprise the same or different adsorbent. The spots are “addressable” in that during mass spectrometry an energy source, such as a laser, may be directed to (i.e., “addresses”) each spot differentially to desorb the analyte.

The probes can be produced using any suitable methods depending on the selection of substrate materials and/or adsorbents. For example, the surface of a metal substrate can be coated with a material that allows derivitization of the metal surface. More specifically, a metal surface can be coated with silicon oxide, titanium oxide or gold. Then surface can be derivatized with a bifunctional linker, one end of which can covalently bind with a functional group on the surface and the other end of which can be further derivatized with groups that function as an adsorbent. In another example, a porous silicon surface generated from crystalline silicon can be chemically modified to include adsorbents for binding markers. In yet another example, adsorbents with a hydrogel backbone can be formed directly on the substrate surface by in situ polymerizing a monomer solution which comprises, e.g., substituted acrylamide monomers, substituted acrylate monomers, or derivatives thereof comprising a functional group of choice as an adsorbent.

The probe substrate comprising an adsorbent contacts a sample. The sample is preferably a biological fluid sample. Examples of biological fluid samples include blood, serum, urine, prostatic fluid, seminal fluid, semen, seminal plasma and prostate tissue (e.g., epithelial tissue, including extracts thereof).

The sample can be solubilized in or admixed with an eluant. The probe substrate comprising an adsorbent then contacts the solution using any techniques including bathing, soaking, dipping, spraying, washing over, or pipetting, etc. Generally, a volume of sample containing from a few attomoles to 100 picomoles of marker in about 1 μl to 500 μl is sufficient for binding to the adsorbent.

The sample can contact the probe substrate comprising an adsorbent for a period of time sufficient to allow the marker to bind to the adsorbent. Typically, the sample and the substrate comprising the adsorbent are contacted for a period of between about 30 seconds and about 12 hours, and preferably, between about 30 seconds and about 15 minutes.

The temperature at which the sample contacts the probe substrate comprising an adsorbent can be a function of the particular sample and the selected probe. Typically, the sample is contacted to the probe substrate under ambient temperature and pressure conditions. For some samples, however, modified temperature (typically 4° C. through 37° C.), and pressure conditions can be desirable, which conditions are determinable by those skilled in the art.

After the probe substrate comprising an adsorbent contacts the sample or sample solution, it is preferred that unbound materials on the probe substrate surface are washed out so that only the bound materials remain on the substrate surface. Washing a probe substrate surface can be accomplished by, e.g., bathing, soaking, dipping, rinsing, spraying, or washing the substrate surface with an eluant or a washing solution. A microfluidics process is preferably used when a washing solution such as an eluant is introduced to small spots of adsorbents on the probe. Typically, the washing solution can be at a temperature of between 0° C. and 100° C., preferably between 4° C. and 37° C.

Any suitable washing solutions or eluants can be used to wash the probe substrate surface. For example, organic solutions or aqueous solutions can be used. Preferably, an aqueous solution is used. Exemplary aqueous solutions include a HEPES buffer, a Tris buffer, a phosphate buffered saline, etc. The selection of a particular washing solution or an eluant is dependent on other experimental conditions (e.g., types of adsorbents used or markers to be detected), and can be determined by those of skill in the art. For example, if a probe comprising a hydrophobic group and a sulfonate group as adsorbents (e.g., SAX1 ProteinChip® array) is used, then an aqueous solution, such as a HEPES buffer, may be preferred. In another example, if a probe comprising a metal binding group as an adsorbent (e.g., Cu(II) ProteinChip® array) is used, then an aqueous solution, such as a phosphate buffered saline, may be preferred. In yet another example, if a probe comprising a hydrophobic group (e.g., H4 ProteinChip® array) is used, then water may be preferred as a washing solution.

An energy absorbing molecule (e.g., in solution) can be applied to markers or other substances bound on the probe substrate surface. Spraying, pipetting, or dipping can be used. This can be done after unbound materials are washed off of the probe substrate surface. An energy absorbing molecule refers to a molecule that absorbs energy from an energy source in a gas phase ion spectrometer, thereby assisting desorption of markers or other substances from a probe surface. Exemplary energy absorbing molecules include cinnamic acid derivatives, sinapinic acid and dihydroxybenzoic acid.

After the marker is bound to the probe, it is detected using gas phase ion spectrometry. Markers or other substances bound to the adsorbents on the probes can be analyzed using a gas phase ion spectrometer. The quantity and characteristics of the marker can be determined using gas phase ion spectrometry. Other substances in addition to the marker of interest can also be detected by gas phase ion spectrometry.

In one embodiment, a mass spectrometer can be used to detect markers on the probe. In a typical mass spectrometer, a probe with a marker is introduced into an inlet system of the mass spectrometer. The analyte is then desorbed by a desorption source such as a laser, fast atom bombardment, or high energy plasma. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of a marker or other substances will typically involve detection of signal intensity. This, in turn, can reflect the quantity and character of a marker bound to the probe.

In a preferred embodiment, a laser desorption time-of-flight mass spectrometer is used with the probe of the present invention. In laser desorption mass spectrometry, a probe with a bound analyte is introduced into an inlet system. The analyte is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio. As any person skilled in the art understands, any of these components of the laser desorption time-of-flight mass spectrometer can be combined with other components described herein in the assembly of mass spectrometer that employs various means of desorption, acceleration, detection, measurement of time, etc.

In another embodiment, an ion mobility spectrometer can be used to detect and characterize a marker. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in the sample. One advantage of ion mobility spectrometry is that it can operate at atmospheric pressure.

In yet another embodiment, a total ion current measuring device can be used to detect and characterize markers. This device can be used when the probe has a surface chemistry that allows only a single type of marker to be bound. When a single type of marker is bound on the probe, the total current generated from the ionized marker reflects the nature of the marker. The total ion current produced by the marker can then be compared to stored total ion current of known compounds. Characteristics of the marker can then be determined.

Data generated by desorption and detection of markers can be analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. Using this information, the program can then identify the set of features on the probe defining certain selectivity characteristics (e.g., types of adsorbent and eluants used). The computer also contains code that receives as input, data on the strength of the signal at various molecular masses received from a particular addressable location on the probe. This data can indicate the number of markers detected, optionally including the strength of the signal and the determined molecular mass for each marker detected.

Data analysis can include the steps of determining signal strength (e.g., height of peaks) of a marker detected and removing “outliers” (data deviating from a predetermined statistical distribution). For example, the observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other substances can be displayed in the form of relative intensities in the scale desired (e.g., 100). Alternatively, a standard may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually.

III. Methods of Detecting Markers

A. Characterization of the Markers

A set of markers was identified that is useful in distinguishing prostate cancer, prostate intraepithelial neoplasia, benign prostate hyperplasia and a negative diagnosis. The set was identified in cell lysate from prostate epithelial tissue.

1. Cell Lysate Marker Set

A set of markers useful for diagnosing prostate cancer, prostate intraepithelial neoplasia, benign prostate hyperplasia and a negative diagnosis has the following molecular weights as determined by mass spectrometry: Marker EP1: 3448±19 Da, Marker EP2: 4036±22 Da, Marker EP3: 4361±24 Da, Marker EP4: 4413±24 Da, Marker EP5: 4639±26 Da, Marker EP6: 4749±26 Da, Marker EP7: 4827±27 Da, Marker EP8: 5666±31 Da, Marker EP9: 8445±46 Da, Marker EP10: 11744±65 Da, Marker EP11: 14696±81 Da, Marker EP12: 24184±133 Da, Marker EP13: 48308±266 Da, and Marker EP14: 53830±296 Da.

These markers are further characterized as follows: Cell lysate from prostate epithelial cells isolated by laser capture microdissection was fractionated and analyzed by retentate chromatography. In this method, test samples were applied to a substrate comprising a copper chelate adsorbent. The adsorbents were washed with a first eluent of 20 mM HEPES, pH 8.0, 0.1% TritonX 100, to allow binding of proteins to the chip, and with a second eluent of phosphate buffered saline (PBS) to remove non-bound materials. Markers retained on the adsorbents were detected by SELDI mass spectrometry. The fourteen markers could be resolved by the metal chelate adsorbent.

B. Sample Sources for Markers

These markers can be detected in many different samples derived from a patient or subject. The sample is preferably a biological fluid sample. Examples of biological fluid samples useful in this invention include blood, serum, urine, prostatic fluid, seminal fluid, semen, seminal plasma and prostate tissue (e.g., epithelial tissue, including extracts thereof).

Because the markers in the Marker Set were discovered in cell lysate from prostate epithelium, this tissue is a preferred sample source for the methods of this invention. More particularly, the samples were collected by laser capture microdissection of this tissue. Therefore, this is a preferred method of obtaining sample for the methods of this invention.

C. Detection of Markers

After a sample is obtained, any suitable method can be used to detect the marker in a sample from a subject being tested. For example, gas phase ion spectrometry or an immunoassay can be used.

1. Mass Spectrometry

In a preferred embodiment, the markers of this invention are detected using mass spectrometry, more preferably using gas phase ion spectrometry and, still more preferably, using surface-enhanced laser desorption/ionization mass spectrometry (“SELDI”). SELDI is an improved method of gas phase ion spectrometry for biomolecules. In SELDI, the surface on which the analyte is applied plays an active role in the analyte capture, desorption and/or desorption.

One popular method of gas phase ion spectrometry for biomolecules is MALDI (matrix-assisted laser desorption/ionization) mass spectrometry. In MALDI, the analyte is typically mixed with a matrix material that, upon drying, forms crystals that capture the analyte. The matrix material absorbs energy from the energy source which otherwise would fragment the bimolecular analytes.

### a) Preparation of Sample

### (i) Pre-Fractionation

In one embodiment, the sample can be pre-fractionated before being subjected to gas phase ion spectrometry. Pre-fractionation has the advantage of providing a less complex sample for analysis. On the other hand, it introduces an extra step in the analytic process that could be unattractive in, for example, a clinical setting. Samples can be pre-fractionated by any means known in the art, including, without limitation, size fractionation and chromatographic fractionation.

In one embodiment, seminal plasma samples can be pre-fractionated before analysis by gas-phase ion spectrometry. A preferred method of fraction includes a first fractionation by gel exclusion chromatography. Sizing columns which exclude molecules whose molecular mass is greater than 30 kDa are particularly useful for this. Fractions of various sizes then can be examined directly or subjected to a second fractionation step based on anion exchange chromatography. Using an anion exchange Q spin column, markers can be eluted using a low strength buffer (e.g., about 10 mM to 50 mM Tris, HEPES or PBS) with salt at low to medium concentration (e.g., about 0.1 M to 0.6 M) and a non-ionic detergent at low concentration (e.g., TritonX 100 at about 0.05 to 0.2%). A particularly useful buffer is 20 mM Tris, 0.5 M NaCl and 0.1% TritonX 100. In another embodiment, the markers are eluted using a pH gradient.

### (ii) Retentate Chromatography

In another embodiment, the sample is fractionated on a bio-chromatographic chip by retentate chromatography before gas phase ion spectrometry. A preferred chip is the Protein Chip® array available from Ciphergen Biosystems, Inc. (Palo Alto, Calif.). As described above, the chip or probe is adapted for use in a mass spectrometer. The chip comprises an adsorbent attached to its surface. This adsorbent can function, in certain applications, as an in situ chromatography resin. In basic operation, the sample is applied to the adsorbent in an eluent solution. Molecules for which the adsorbent has affinity under the wash condition bind to the adsorbent. Molecules that do not bind to the adsorbent are removed with the wash. The adsorbent can be further washed under various levels of stringency so that analytes are retained or eluted to an appropriate level for analysis. Then, an energy absorbing molecule can be added to the adsorbent spot to further facilitate desorption and ionization. The analyte is detected by desorption from the adsorbent, ionization and direct detection by a detector. Thus, retentate chromatography differs from traditional chromatography in that the analyte retained by the affinity material is detected, whereas in traditional chromatography, material that is eluted from the affinity material is detected.

A useful adsorbent for the markers of the Marker Set is a metal chelate adsorbent and, in particular, copper. This surface also is usefully washed with a pH neutral buffer such as PBS. A preferred surface is IMAC3 from Ciphergen Biosystems, Inc. IMAC3 comprises a copper chelate adsorbent.

Another useful adsorbent for any of the markers of this invention is an antibody that specifically binds the marker. Chips comprising antibodies that bind to one or more markers are particularly useful for removing non-markers which do not bind to the antibodies and which function as “noise” in the detection process.

As will be evident to anyone skilled in the art, different markers may be more easily resolved using different combinations of adsorbents and eluants.

### (iii) Mixing the Sample with an Energy Absorbing Matrix

In MALDI applications the sample to be analyzed is mixed with an energy absorbing matrix prior to ionization and mass analysis. The sample/matrix mixture is then applied to the surface of an inert mass spectrometer probe. Suitable matrix materials are well know to those of skill in the art and include 3-hydroxypicolinic acid (3-hydroxy-2-pyridinecarboxylic acid), nicotinic acid, N-oxide, 2′-6′-dihydroxyacetophenone, gentisic acid (2,5-dihydroxybenzoic acid), a-cyano-4-hydroxycinnamic acid, ferulic acid (4-hydroxy-3-methoxycinnamic acid) and sinapinic acid (3,5-dimethoxy-4-hydroxycinnamic acid).

The resolving power of MALDI is limited by the complexity of the sample being analyzed. Therefore prefractionation or otherwise purifying the sample prior to MALDI analysis is preferred.

### (iv) Modification of Marker Before Analysis

In another embodiment, the markers are modified before detection in order to alter their molecular weight. These methods may decrease ambiguity of detection. For example, the markers may be subject to proteolytic digestion before analysis. Any protease can be used. Proteases such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry.

In another embodiment, the markers can be modified by the attachment of a tag of particular molecular weight that bind specifically to molecular markers, further distinguishing them.

### b) Performance of Laser Desorption/Ionization Mass Spectrometry

After the marker is detected by mass spectrometry, preferably gas phase ion spectrometry, a test amount of marker can be determined. For example, a signal is displayed at the molecular weight of the marker of interest. Based on the strength or magnitude of the displayed signal, the amount of marker in a sample being tested can be determined. It is noted that the test amount of marker in a sample need not be measured in absolute units, but can be in relative units as long as it can be compared qualitatively or quantitatively to a control amount of a marker. For example, the amount of the marker detected can be displayed in terms of relative intensity based on the background noise. Preferably, the test amount and the control amount of markers are measured under the same conditions.

If desired, the absolute amount of a marker can be determined by calibration. For example, a purified, known marker can be added in increasing amounts to different spots of adsorbents on the probe surface. Then peaks from each spot can be obtained and plotted in a graph against the concentration of known marker protein at each spot. From the peak intensity vs. concentration plot, the absolute amount of a marker in any sample being tested can be determined.

2. Immunoassay Detection

In another embodiment of the detection method, an immunoassay can be used to qualitatively or quantitatively detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 2nd Ed. (Sambrook et al., Cold Spring Harbor Laboratory, NY 1989).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Terr, eds., 7th ed. 1991).

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. (See e.g. Xiao et al., Cancer Research 62: 6029-6033 (2001)) The sample is preferably a biological fluid sample taken from a subject. Examples of biological fluid samples include blood, serum, urine, prostatic fluid, seminal fluid, semen, seminal plasma and prostate tissue (e.g., epithelial tissue, including extracts thereof). In a preferred embodiment, the biological fluid comprises seminal plasma The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay techniques are well-known in the art, and a general overview of the applicable technology can be found in Harlow & Lane, supra.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. As noted above, the test amount of marker need not be measured in absolute units, as long as the unit of measurement can be compared to a control amount.

IV. Methods of Diagnosis Using Markers

The markers in the Marker Set may be used alone, in combination with other markers in the set, or with other markers entirely (e.g., PSA or prostate specific membrane antigen) to aid in the diagnosis of prostate cancer, prostate intraepithelial neoplasia, benign prostate hyperplasia or a negative diagnosis. Used together, they tend to provide more information for the diagnostician, increasing the percentage of true positive and true negative diagnoses and decreasing the percentage of false positive or false negative diagnoses, than a single marker alone.

In certain embodiments, it is useful to detect the presence or absence of a marker, without quantifying the amount of marker. This information is useful because the markers show differences in frequency of detection between PCA, PIN, BPH and negative diagnosis. In other embodiments, detecting the presence or absence of the markers can also involve quantifying the markers. This information is useful for several purposes. First, individual markers may be slightly detectable in certain states based on a weak peak, but easily detectable in other states based on a strong peak. Second, certain of the markers are frequently detected in all states, but their relative ratio differs dramatically among PCA PIN, BPH or normal samples.

It is, of course, understood by diagnosticians that diagnostic tests are measured by their degree of specificity and sensitivity. Most diagnostic tests suffer from some imperfection in their ability to provide a positive result for every true positive diagnosis, as well as some imperfection in their ability to provide a negative result for every true negative diagnosis. However, tests which are not perfectly specific or sensitive are useful in diagnosis because they render certain diagnoses more likely than not. For example, markers EP5 and EP12 are detected in 43% of all samples positive for PCA and 0% of negative samples. Thus, detection of EP5 or EP12 is useful in that it provides a probable diagnosis of PCA. Failure to detect EP5 or EP12 also is useful in that it provides a probable negative diagnosis. Furthermore, used in combination with other markers, the diagnostician can increase their confidence in a PCA or negative diagnosis. With this in mind, the markers have frequency distributions as follows.

A. Cell Lysate Marker Set

Table 1 is a comparison of the relative abundance levels of markers EP1 to EP14 in BPH, PIN, and PCA cell lysates. The relative quantity can be determined in gas phase ion spectrometry either by peak height or peak area. Preferably, this determination is normalized with respect to an internal standard, which is present in all samples. Markers EP2, EP3, EP5, and EP6 occur in a higher abundance in both PIN and PCA samples. Therefore, detection of two or three of these markers is positively correlated with a pathologic diagnosis. EP8 occurs in higher abundance in BPH samples. Therefore, detection of this marker is positively correlated with a normal or benign diagnosis.

Table 2 displays the percent of BPH, PIN, or PCA prostate cell lysates found to be over- or under-expressing markers EP1-EP14. Marker EP1 appears to be expressed in all cell types but is present in increasing amounts in BPH, PIN, and PCA cell lysates. EP10 appears to be under-expressed in BPH and PIN cell lysates and a majority of the PCA cell lysates. EP13 appears to be under-expressed in a majority of the PIN and PCA cell lysates.

Table 3 shows the statistical analysis of the differential expression of selected proteins as determined by the Wilcoxon signed ranks test. The differential expression of EP2, EP3, and EP6 was significant in differentiating PIN and/or PCA from non-cancer cells; while the differential expression of EP8 was significant in distinguishing BPH from PIN and PCA.

Table 4 displays the sensitivity and specificity of single markers and combination of markers found in PIN and PCA cell lysates. Combining markers EP2 and EP5, EP3 and EP5, or EP3 and EP6 increased the sensitivity for identifying PIN or PCA samples. Therefore, detection of these combinations of markers is positively correlated with a pathological diagnosis.

Table 5 shows that by combining the expression levels of seven biomarkers, EP2, EP3, EP4, EP5, EP6, EP8, and PSA, a specificity of 93.3% and sensitivity of 93.8%, with an overall accuracy of 93.5% was achieved in differentiating normal (N/BPH) from cancer (PIN/PCalif.).

B. Detection of Markers: Comparison with Controls

Detection of markers can usefully involve comparing the test data to a control. The control can be useful for calibration of “normal” amounts and for quantifying the amount of a marker against a normal or control amount. For example, it can be useful to know whether a marker present in both normal and pathologic conditions is increased by 1.5 fold, by 2 fold, by 5 fold, or by 10 fold compared to the control amount.

In one embodiment, the control amount can be an amount of a marker present in comparable samples from normal patients. The control amount is measured under the same or substantially similar experimental conditions as in measuring the test amount. For example, if a test sample is obtained from a subject's seminal plasma and a marker is detected using a particular probe, then a control amount of the marker is preferably determined from a seminal plasma sample of a patient using the same probe. It is preferred that the control amount of marker is determined based upon a significant number of samples from subjects who do not have prostate cancer (e.g., BPH or negative diagnosis patients) so that it reflects variations of the marker amounts in that population.

C. Correlation By Computer

Data generated by mass spectrometry is usefully analyzed by computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal,” “BPH” and “PCA” and determines the closeness of fit between the two signals. The software also can include code indicating which the test sample is closest to, thereby providing a probable diagnosis. The software can also include code that executes an algorithm which comprises an artificial intelligence program. Examples of artificial intelligence programs include fuzzy logic, cluster analysis or neural networks. Executing the artificial intelligence program can include code indicating that the test sample correlates most closely with a normal, BPH, PIN or PCA sample, thereby providing a probable diagnosis.

Data generation in mass spectrometry begins with the detection of ions by an ion detector. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants. Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that 30 represent signal from an analyte. Peak selection can, of course, be done by eye.

However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “rain” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, Jan. 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are in U.S. Provisional Patent Application Nos. 60/249,835, filed on Nov. 16, 2000, and 60/254,746, filed on Dec. 11, 2000, and U.S. Non-Provisional Patent Application Nos. 09/999,081, filed Nov. 15, 2001, and 10/084,587, filed on Feb. 25, 2002. All of these U.S. Provisional and Non Provisional Patent Applications are herein incorporated by reference in their entirety for all purposes.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Using pattern recognition techniques discussed above, classification algorithms may be developed from the classification models. These are highly useful in aiding the diagnosis of prostate cancer, prostate intraepithelial neoplasia, or benign prostate hyperplasia versus a negative diagnosis, using the markers of the present invention. By screening samples from sources known to have expressed each condition, the training data set, the pattern recognition techniques described herein identify difference patterns in the marker composition of diseased tissue relative to normal tissue. These difference patterns may comprise differences in the presence and/or amount of a particular marker or markers. Difference patterns may be built from a statistical analysis of several test data sets using methods well known to those of skill in the art. The difference patterns generated may then be used to construct classification models, as described above, and used in comparison studies to diagnose disease states of patients.

V. Kits

In yet another aspect, the invention provides kits for aiding a diagnosis of prostate cancer, wherein the kits can be used to detect the markers of the present invention. For example, the kits can be used to detect any one or combination of markers described above, which markers are differentially present in samples of a prostate cancer patient, BPH and normal patients. The kits of the invention have many applications. For example, the kits can be used to differentiate if a subject has prostate cancer, BPH or has a negative diagnosis, thus aiding a prostate cancer diagnosis. In another example, the kits can be used to identify compounds that modulate expression of the markers in in vitro prostate cells or in vivo animal models for prostate cancer.

In one embodiment, a kit comprises: (a) a substrate comprising an adsorbent thereon, wherein the adsorbent is suitable for binding a marker, and (b) a washing solution or instructions for making a washing solution, wherein the combination of the adsorbent and the washing solution allows detection of the marker using gas phase ion spectrometry. Such kits can be prepared from the materials described above, and the previous discussion of these materials (e.g., probe substrates, adsorbents, washing solutions, etc.) is fully applicable to this section and need not be repeated.

In some embodiments, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate.

Optionally, the kit may further comprise instructions for suitable operational parameters in the form of a label or a separate insert. For example, the kit may have standard instructions informing a consumer how to wash the probe after a sample of seminal plasma is contacted on the probe.

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and need not be repeated.

In either embodiment, the kit may optionally further comprise a standard or control information so that the test sample can be compared with the control information standard to determine if the test amount of a marker detected in a sample is a diagnostic amount consistent with a diagnosis of prostate cancer.

### EXAMPLES

The following examples show the identification of markers that have positive or negative correlations with prostate cancer, prostate intraepithelial neoplasia, or benign prostate hyperplasia. Markers were determined from samples of cell lysates isolated by laser capture microdissection.

VI. Tissue Specimens

Prostate tissues were procured from consenting patients undergoing radical prostatectomy, following approved protocols. The tissues were processed immediately and stored in the Virginia Prostate Center's Bio-repository. Tissue pieces harvested for LCM were immediately embedded in OCT and stored at −80° C. One cryosection was stained with H&E and examined by a pathologist to identify cells of interest. Mirrored H&E stained sections, fixed in formalin and paraffin embedded, were also used to further aid in the identification of specific cell types. Additional serial frozen sections at 8 microns were used for microdissection.

VII. Laser Capture Microdissection

Populations of normal luminal epithelia, Benign Prostatic Hyperplasia (BPH), Prostate Intraepithelial Neoplasia (PIN), and Prostate Adenocarcinoma (PCA) epithelial cells were microdissected from frozen tissue sections using the PixCell™ II Laser Capture Microdissection Microscope (Arcturus Engineering, Inc., Mountain View, Calif.) essentially as described by Emmert-Buck et al., Science, 274: 998-1001 (1996). See also U.S. Pat. No. 5,843,644 (Liotta et al.) and U.S. Pat. No. 5,843,657 (Liotta et al.). The procedure for staining frozen sections for LCM was followed with slight modifications: the hematoxylin step was omitted and protease inhibitors (Complete, Roche Biomedical, Indianapolis, Ind.) were added to the staining baths. A total of 1000 laser shots totaling 3000-6000 cells were procured for each cell type. Matched benign and diseased epithelial cell types were harvested from each prostate sample. In some cases, adjacent stroma cells were also procured. Samples were standardized by total number of laser shots, and duplicate samples were captured from the same areas of each serial section to check reproducibility.

VIII. Laser Desorption/Ionization Mass Spectrometry

Cell lysates were immediately prepared after microdissection by adding 4 μl of the lysis buffer containing 20 mM HEPES pH 8.0 with 1% Triton X-100 directly on the LCM cap. Each lysate was diluted 1:10 in PBS buffer, giving a total volume of 40 ul. Lysates were vortexed for 10 minutes at 4° C. and centrifuged briefly to remove cellular debris. The supernatant was added to an IMAC3 (Immobilized Metal Affinity Capture) ProteinChip® Array (Ciphergen Biosystems Inc. Fremont, Calif.), pretreated with 100 mM CuSO₄, following the manufacturer's instructions. A bioprocessor (Ciphergen Biosystems) was fitted on top of the chip arrays to permit the addition of the 40 ul sample. To control for variation, cell lysates harvested from each prostate tissue were analyzed on a single Biochip. The array was then incubated with the diluted lysate overnight with continuous shaking. After removal of the lysate, each spot was washed twice with PBS followed by a final water rinse.

The arrays were allowed to air dry and a saturated solution of sinapinic acid (Ciphergen Biosystems) in 50% (v/v) acetonitrile, 0.5% (v/v) trifluoroacetic acid was added to each spot. Time of flight mass spectra were generated in a Ciphergen Protein Biology System II (PBS II) by averaging 120 laser shots collected in the positive mode at laser settings of 225 and 280. Data was calibrated externally using purified peptide and protein standards.

Spectra were analyzed with the Ciphergen Peaks 2.1 software and relative abundances for each peak were calculated as follows. The relative abundance of the proteins were subdivided into three classes: Low (+) equates to 1-30% of spectral scale, Medium (++) equates to 31-60%, and High (+++) equates to 60-100%. Numerical values were then assigned to the abundance levels (i.e. (+) equates to 33, (++) equates to 66, (+++) equates to 100) and averaged for each cell type for the purpose of representing the protein expression between prostate cell types in graphical form.

Sensitivity is defined as the percent of diseased (BPH/PIN/PCA) cell types for which the biomarker of interest is present, (true positive/total number of diseased lysates×100). Specificity is defined as the percent of cell types for which the biomarker of interest is not positive, (true negative/total number of lysates without disease×100).

IX. Markers in Cell Lysates from Prostate Epithelial Tissue

A. IMAC3 ProteinChip
1. Protocol:
1. Draw a hydrophobic ring around each adsorbent spot on the chip.
2. Load 10 μl 100 mM CuSO₄. Incubate chip for 10 minutes in a moist chamber with shaking.
3. Wash Chip with DI H₂O 10 seconds, around spots and remove excess water.
4. Load 10 μl 100 mM sodium acetate, pH 4.0. Incubate chip for 10 minutes in a moist chamber with shaking.
5. Wash Chip with DI H₂O and allow to air dry.
6. Lyse microdissected cells in 4 μl of 20 mM HEPES 1% Triton X-100 directly on the cap. Pipette the lysis buffer up and down on top of the cells until lysis occurs. Dilute lysate 1:10 in PBS and vortex cell lysate 10 minutes at 4° C. and spin down.
7. Spot the entire lysate (40 μl) on spot and incubate in a moist chamber overnight with continuous shaking.
8. Wash each spot 2× with 40 μl of PBS.
9. Wash 1×40 μl with water and air dry.
10. Spot 2×0.5 μl sinapinic acid in 50% acetonitrile, 0.5% trifluoroacetic acid.
11. Air dry.

2. SELDI Reader Data Collection
1. Peaks were collected on the Ciphergen Biosystems PBSII at a laser intensity of 225 and 280 with a detector sensitivity of 10. 120 shots were collected and averaged.
2. Data was calibrated externally using purified peptide and protein standards.

B. Results

Pure populations of matched benign (normal or BPH), PIN (high grade) and/or PCA epithelial cells were obtained from nine prostatectomy specimens examined. Stroma cells were also microdissected from a selected subset of tissues to compare to the epithelial cell profiles. A total of 80 cell lysates from an average of 5000 cells/protein array were analyzed by SELDI time-of flight mass spectrometry. Processing the lysates on an immobilized metal affinity capture surface pre-treated with CuSO₄ resolved an average of 70 protein or peptide peaks in the range from 3 kDa to 100 kDa.

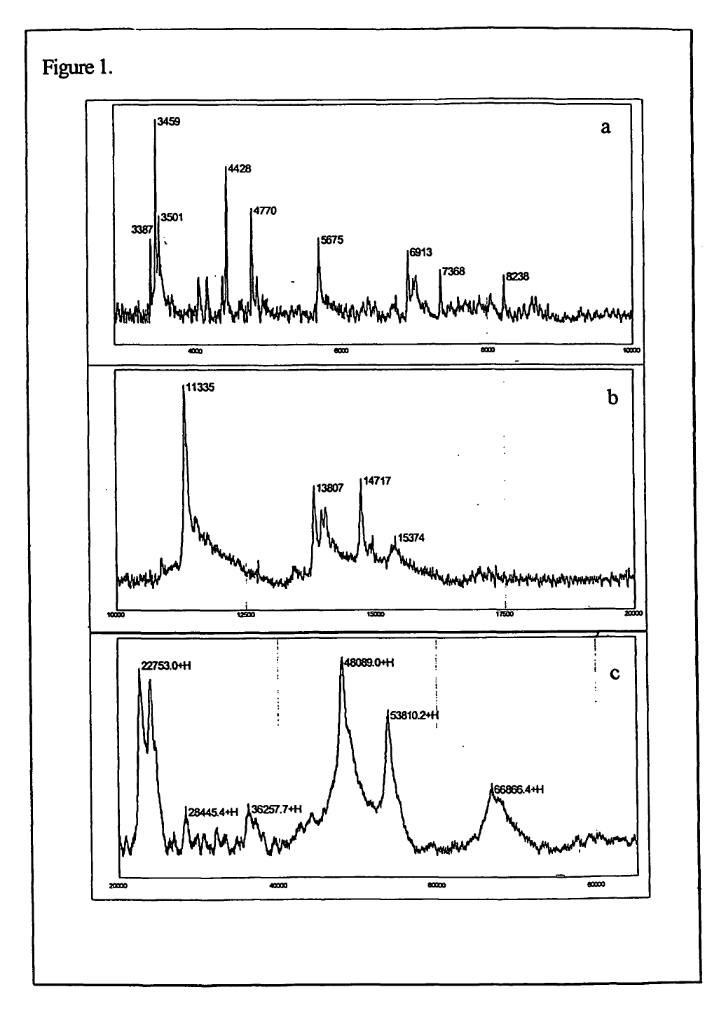

FIG. 1 is a representative spectrum of the protein profile of a PCA cell lysate in the mass range of 3000-80,000 Da. The advantages of the SELDI technology in resolving small molecular weight protein or peptide species is evident, since the majority of peaks observed were below 20 kDa. FIG. 1a shows an abundant set of peaks observed as a triplet, with the predominant peak at m/z of 3459. This cluster was observed in most cell lysates analyzed, stromal and epithelial, and has previously been identified in our laboratory as defensin α-1 from the urine of bladder cancer patients using a SELDI immunoassay (see e.g. Vlahou et al., Am. J Pathol., 158: 1491-1502, (2001)). A peak at m/z of 28445 (FIG. 1c) is most likely free PSA. Serum albumin (HSA), a peak at m/z of 66866 was also present in all cell lysates tested (see FIG. 1c).

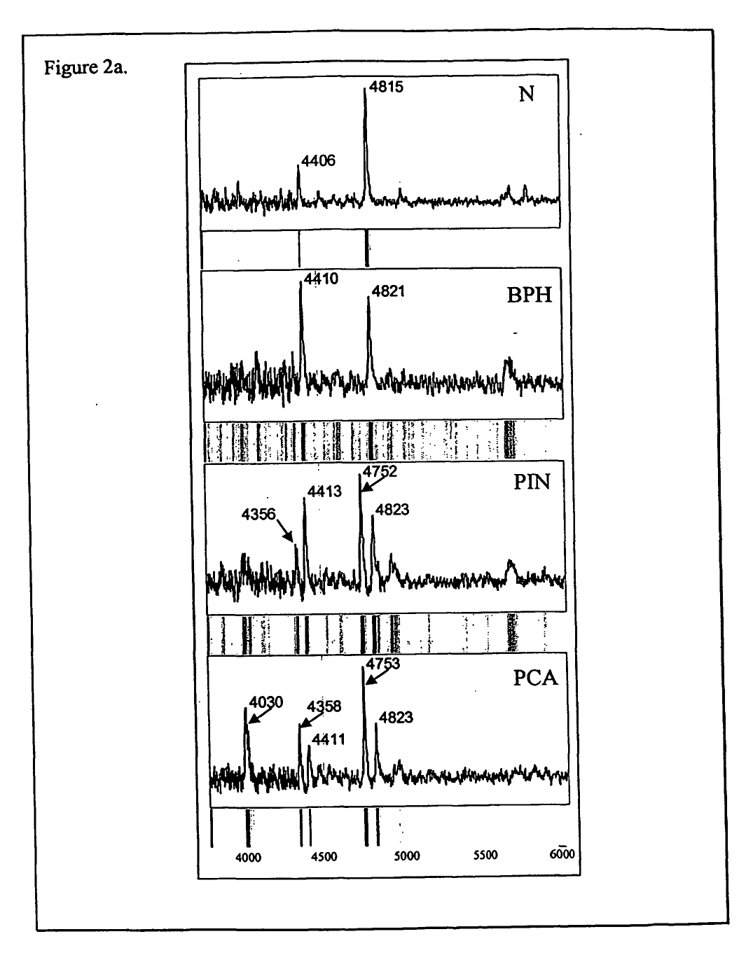

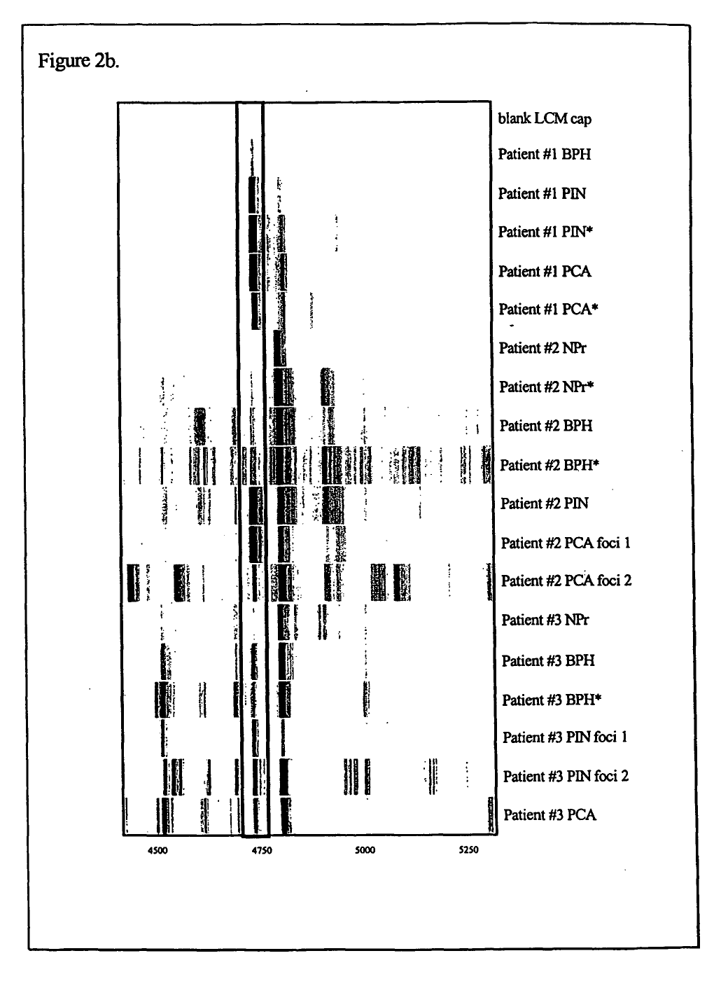

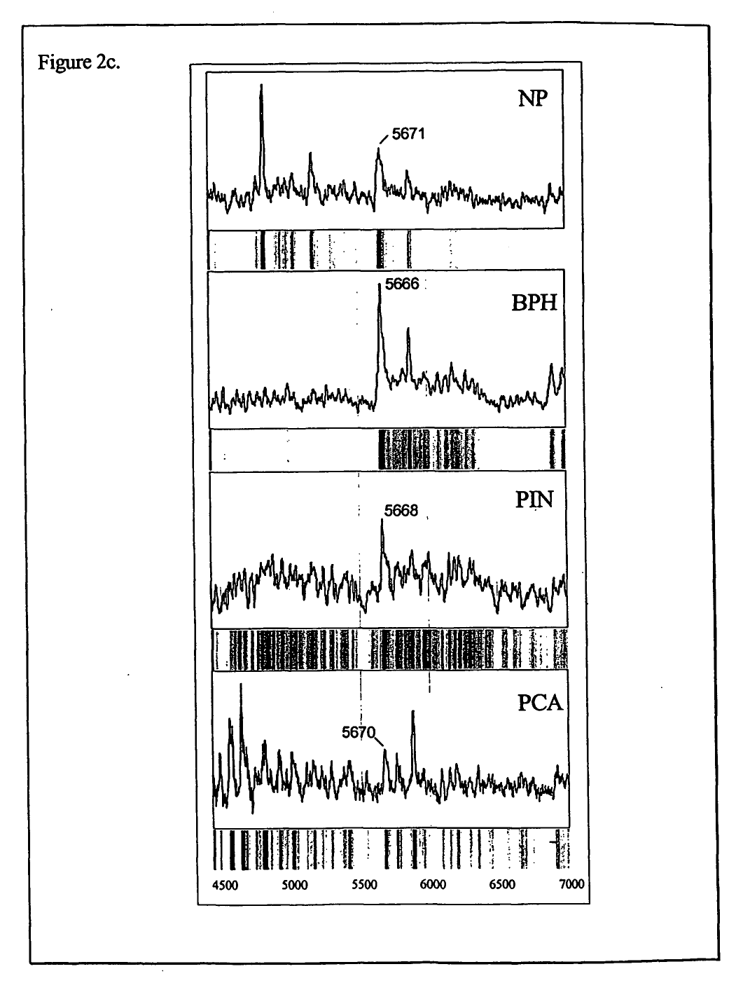

Differential protein expression between cell types was evaluated by determining the abundance (i.e., over-expression or under-expression) of each peak relative to each matched cell type. FIG. 2a is a an example of a group of peaks between 4000 Da and 6000 Da showing differential expression between the epithelial cell types obtained from the same prostate tissue specimen. Three of these peaks (m/z 4030 (EP2), 4358 (EP3), 4753 (EP6)) occur in greater frequency and abundance in the PIN and PCA lysates. As a general rule, peptide or protein species found in greater abundance in the PCA profiles, were also found in increasing amounts in the PIN profiles. FIG. 2b shows the over-expression of the peak with an average m/z of 4749 (EP6) in the PIN and PCA lysates from three different prostatectomy specimens. Duplicate samples exhibited a high degree of reproducibility. In contrast, regions of heterogeneity were present in the protein profiles derived from two different foci of PCA in Patient #2 and two foci of PIN in Patient #3. Profile differences were also found in the BPH cell lysates. FIG. 2c is an example of a peak at m/z of 5666 (EP8), which appears in greater abundance in the BPH epithelial cells when compared to the spectra of the other matched cell types. With few exceptions, the protein patterns found in BPH cell lysates more closely resembled the normal epithelial profile.

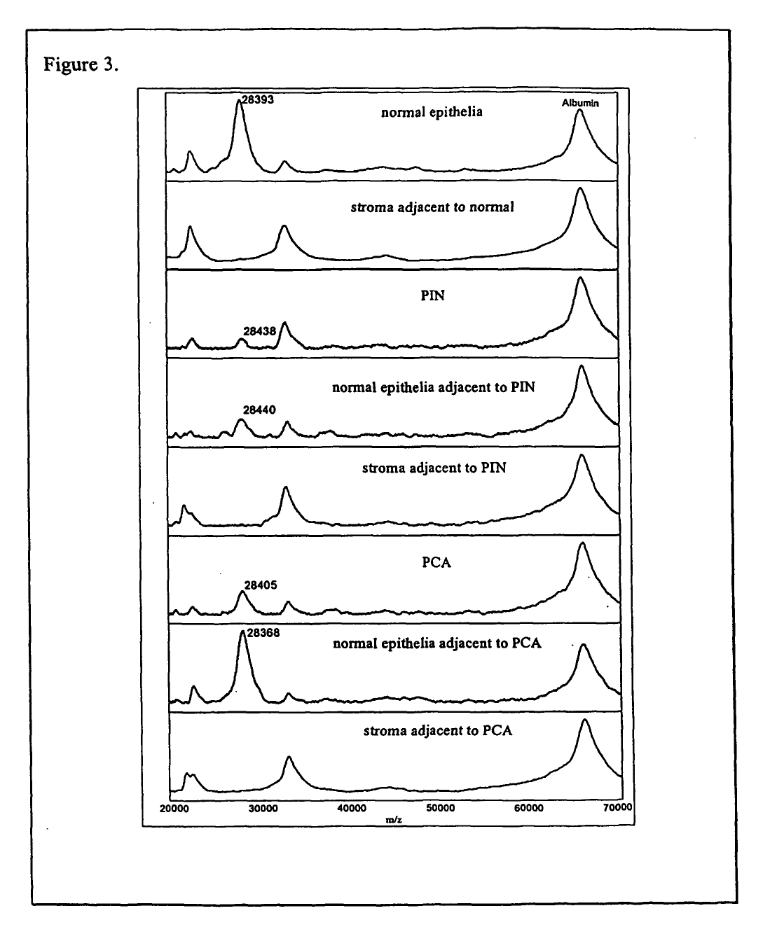

The profiles of proteins greater than 10 kDa also demonstrated some differential expression patterns. One peak with an average m/z 28442 was found in lower abundance in the BPH, PIN and PCA epithelial cells compared to profiles of matched normal cell lysates. This mass is consistent with the molecular weight of free PSA (Belanger et al., Prostate 27:187-197 (1995)). An example of this decrease in intracellular PSA levels for PIN and PCA cells can be seen in FIG. 3. A large PSA peak at 28393 Da was present in the normal prostate epithelia but absent from adjacent stroma cells. The PIN and PCA cells have a greatly reduced PSA peak. Normal epithelial cells procured directly adjacent to the PCA foci also exhibited a large amount of intracellular PSA. The 66838 Da peak is albumin and was present in all cell lysates at high abundance.

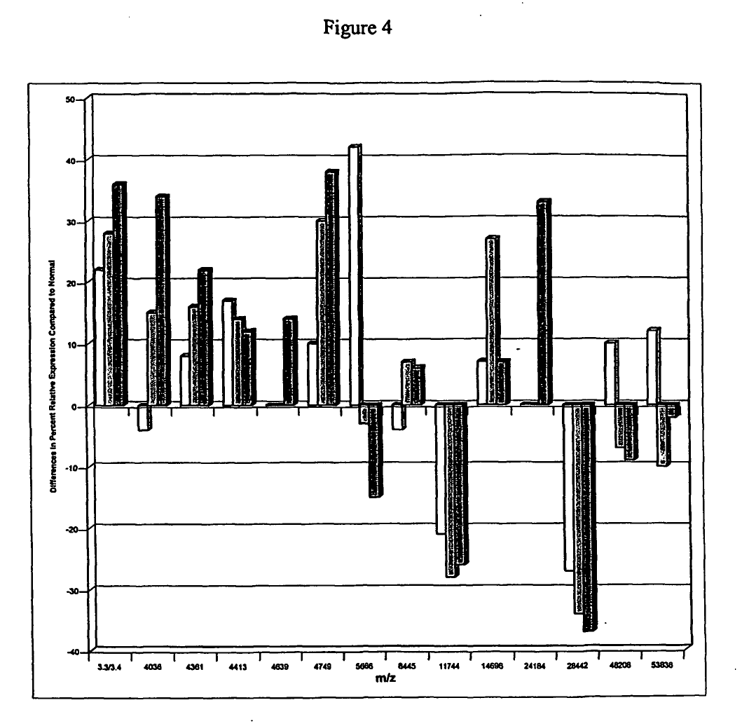

These comparative analyses suggested that several proteins showed differential expression between the four cell types. However, to more precisely select those with high biomarker potential, it was necessary to standardize peak expression levels throughout the matched populations. The relative abundance of the proteins were subdivided into three classes: Low (+) equates to 1-33% of spectral scale, Medium (++) equates to 33-66%, and High (+++) equates to 66-100%. Each individual spectrum was expanded in specific mass ranges to assist in determining the relative abundance of each observed peak. An in-house computer program was used to cluster the peaks to obtain an average mass for each peak with a mass error range of 0.15%. From an average of 70 peaks observed, 14 (20%) displayed expression differences between the epithelial cell profiles (Table 1). The scores in Table 1 were converted to numerical values and plotted in a histogram showing the average differences in expression levels between cell types subtracted from the expression level in normal epithelia (FIG. 4). The over-expression and under-expression for these 14 protein/peptides was “normalized” and evaluated for BPH, PIN and PCA cells. Generally, the PCA and PIN levels of expression for protein or peptides were similar. As shown in FIG. 4, there were higher levels of a group of small molecular weight peptides/proteins between 3000 and 6000 Da in the PIN and PCA profiles. The first of these, 3448 Da, was found increased in BPH, PIN, and PCA cell lysates. Three peaks (4036 Da (EP2), 4361 Da (EP3), and 4749 Da (EP6)) were over-expressed in PIN and PCA with the highest level of over-expression in PCA lysates. Note that none of the BPH cell lysates exhibited the 4036 Da (EP2) peak. A summary of the percent of each cell type exhibiting those most significantly increased or decreased proteins is presented in Table 2.

TABLE 1
Comparison of the abundance levels* of proteins in lysates
of LCM procured prostate cells (epithelial and stromal).

| | mw(Da) | | | | | | | | | | | | | | |
|---|---|---|---|---|---|---|---|---|---|---|---|---|---|---|---|
| specimen # | 3448 | 4036 | 4361 | 4413 | 4639 | 4749 | 4827 | 5666 | 8445 | 11744 | 14696 | 24184 | 28422 | 48308 | 53830 |

CA3491

| | | | | | | | | | | | | | | | |
|---|---|---|---|---|---|---|---|---|---|---|---|---|---|---|---|
| BPH | + | 0 | 0 | 0 | 0 | + | + | +++ | 0 | 0 | + | 0 | + | ++ | + |
| PIN | ++ | + | 0 | ++ | 0 | ++ | + | 0 | 0 | 0 | +++ | 0 | ++ | + | 0 |
| PCA | +++ | + | 0 | ++ | 0 | +++ | ++ | ++ | 0 | + | ++ | +++ | + | +++ | ++ |
| CA3495 | | | | | | | | | | | | | | | |
| N | ++ | + | + | + | 0 | 0 | ++ | + | 0 | + | +++ | 0 | ++ | + | + |
| BPH | +++ | 0 | 0 | ++ | 0 | 0 | ++ | ++ | 0 | + | ++ | 0 | + | ++ | ++ |
| PIN | +++ | + | + | ++ | 0 | ++ | + | + | 0 | ++ | ++ | 0 | ++ | +++ | + |
| PCA | +++ | ++ | ++ | + | 0 | +++ | ++ | 0 | 0 | + | ++ | +++ | + | + | + |
| CA3501 | | | | | | | | | | | | | | | |
| N | +++ | 0 | ++ | + | 0 | 0 | 0 | 0 | 0 | ++ | + | 0 | ++ | ++ | + |
| BPH | +++ | 0 | ++ | + | 0 | + | ++ | 0 | 0 | ++ | ++ | 0 | + | 0 | 0 |
| PIN | +++ | + | ++ | + | 0 | + | ++ | 0 | 0 | ++ | ++ | 0 | ++ | ++ | + |
| CA3510 | | | | | | | | | | | | | | | |
| N | +++ | 0 | 0 | 0 | 0 | 0 | 0 | 0 | + | ++ | 0 | 0 | +++ | 0 | 0 |
| PIN | +++ | 0 | 0 | 0 | 0 | + | + | 0 | 0 | 0 | +++ | 0 | ++ | 0 | 0 |
| PCA | +++ | 0 | 0 | 0 | 0 | + | + | 0 | 0 | 0 | 0 | + | +++ | 0 | 0 |
| CA3517 | | | | | | | | | | | | | | | |
| N | ++ | 0 | + | + | 0 | 0 | + | + | 0 | +++ | 0 | 0 | +++ | + | 0 |
| BPH | ++ | 0 | ++ | ++ | 0 | 0 | + | ++ | 0 | + | 0 | 0 | +++ | ++ | + |
| PIN | + | 0 | ++ | + | 0 | + | ++ | + | 0 | 0 | 0 | 0 | 0 | 0 | 0 |
| PCA | +++ | + | ++ | + | + | 0 | +++ | 0 | 0 | 0 | 0 | 0 | 0 | 0 | 0 |
| CA3511 | | | | | | | | | | | | | | | |
| N | + | 0 | 0 | 0 | 0 | 0 | +++ | + | 0 | + | 0 | 0 | ++ | ++ | + |
| BPH | ++ | 0 | + | + | 0 | 0 | +++ | +++ | 0 | 0 | 0 | 0 | ++ | + | + |
| PIN | ++ | + | ++ | + | 0 | + | +++ | + | 0 | 0 | + | 0 | ++ | + | + |
| PCA | + | +++ | ++ | + | + | + | ++ | + | + | + | 0 | 0 | ++ | + | + |
| CA3535 | | | | | | | | | | | | | | | |
| N | 0 | 0 | 0 | 0 | 0 | 0 | + | +++ | 0 | + | 0 | 0 | +++ | + | + |
| PIN | ++ | 0 | 0 | 0 | 0 | 0 | + | + | ++ | + | 0 | 0 | + | 0 | 0 |
| PCA | ++ | 0 | + | 0 | + | 0 | ++ | 0 | + | + | 0 | 0 | + | 0 | 0 |
| CA3539 | | | | | | | | | | | | | | | |
| N | 0 | 0 | + | 0 | 0 | 0 | ++ | 0 | 0 | + | 0 | 0 | + | 0 | 0 |
| BPH | ++ | 0 | + | 0 | 0 | 0 | 0 | ++ | 0 | + | 0 | 0 | 0 | + | + |
| PIN | +++ | 0 | ++ | 0 | 0 | 0 | +++ | + | 0 | + | 0 | 0 | 0 | 0 | 0 |
| CA3546 | | | | | | | | | | | | | | | |
| N | + | 0 | 0 | 0 | 0 | 0 | ++ | + | 0 | + | 0 | 0 | ++ | + | + |
| BPH | ++ | 0 | 0 | 0 | 0 | 0 | 0 | +++ | 0 | + | 0 | 0 | ++ | + | + |
| PIN | ++ | + | + | 0 | 0 | 0 | 0 | ++ | + | 0 | + | 0 | 0 | 0 | 0 |
| PCA | +++ | + | ++ | 0 | 0 | 0 | + | 0 | 0 | + | + | 0 | 0 | 0 | 0 |
| stroma adj. | | | | | | | | | | | | | | | |
| to | + | 0 | + | 0 | 0 | 0 | ++ | 0 | + | 0 | 0 | 0 | 0 | 0 | 0 |
| to PIN/ | ++ | 0 | ++ | 0 | 0 | 0 | ++ | 0 | + | 0 | 0 | 0 | 0 | 0 | 0 |

*Relative abundance of the proteins: + = 1-33% of spectral scale, ++ = 33-66%, +++ = 66-100%, 0 = peak not detected.

Two peaks (4639 Da (EP5) and 24184 Da (EP12)) were specific to PCA lysates in 43% of the PCA samples tested. The peak at 4749 Da (EP6) was over-expressed in 67% and 57% of PIN and PCA lysates, respectively, with the highest overall peak expression observed in the PCA lysates. A 14696 Da (EP11) peak was present in 56% of the PIN cell lysates. Also of note, was a large increase in expression of a peak at 5666 Da (EP8) found in 86% of the BPH profiles. A peak at m/z 4413 (EP4) and two high molecular weight proteins (48208 Da (EP13) and 53836 Da (EP14)) were also found to have increased expression in BPH cells. As shown in FIG. 4, a few proteins were under-expressed in BPH, PIN, and PCA cells where compared to the expression levels in normal epithelial cells. Expression of the 5666 Da (EP8) peak, which was over-expressed in BPH profile, was reduced in 22% of PIN ad 71% of PCA cell lysates at significant levels (Table 2). In addition, decreased expression of an 11744 Da protein (EP10) and PSA (28422 Da) was found in 56% and 57% of the PIN and PCA cell lysates. The reduced expression of intracellular PSA was found in 56% and 57% of PIN and PCA cell lysates respectively.

TABLE 2
Percent of prostate cells lysates (BPH/PIN/PCA) showing differential
expression of selected proteins (i.e. greater or lesser abundance) when
compared to the expression in matched normal prostate epithelial cell lysates.

| | BPH | | PIN | | PCA | |
|---|---|---|---|---|---|---|
| | % of sample | | % of sample | | % of sample | |
| | | Lesser | Greater | Lesser | Greater | Lesser |
| average m/z | Greater abundance | abundance | abundance | abundance | abundance | abundance |
| 3448 | 57 | 0 | 67 | 11 | 71 | 0 |
| 4036 | 0 | 0 | 44 | 0 | 71 | 0 |
| 4361 | 29 | 14 | 44 | 0 | 71 | 0 |
| 4413 | 43 | 0 | 33 | 0 | 29 | 14 |
| 4639 | 0 | 0 | 0 | 0 | 43 | 0 |
| 4749 | 14 | 0 | 67 | 0 | 57 | 0 |
| 5666 | 86 | 0 | 22 | 22 | 0 | 71 |
| 8445 | 0 | 0 | 22 | 11 | 29 | 0 |
| 11744 | 0 | 43 | 11 | 44 | 14 | 29 |
| 14696 | 14 | 14 | 56 | 0 | 29 | 14 |
| 24184 | 0 | 0 | 0 | 0 | 43 | 0 |
| 28422 | 0 | 43 | 11 | 56 | 0 | 57 |
| 48308 | 57 | 43 | 11 | 56 | 14 | 43 |
| 53830 | 29 | 14 | 0 | 33 | 14 | 14 |

The significance of the expression differences between cell types was evaluated using the Wilcoxon signed rank test (Table 3). Expression differences were found to be significant (p<0.05) for BPH vs. PIN and BPH vs. PCA for over-expression of the 4036 Da (EP2) protein (Table 3). A comparison of N vs. PCA approaches significance (p=0.059) for this peak. Similarly, significant differences were observed in N vs. PCA, and BPH vs. PIN in over-expression of the 4361 (EP3) peak. Over-expression of the 4749 Da (EP6) peak was significant for N vs. PIN and approaches significance for BPH vs. PIN (Table 3). The 5666 Da (EP8) protein was significantly over-expressed in N vs. BPH and, interestingly, is significantly under-expressed in BPH vs. PIN and BPH vs. PCA. The under-expression of PSA was significant in N vs. PIN and approaches significance (p=0.066) for N vs. PCA.

TABLE 3
Wilcoxon signed ranks test of peak expression differences.

| | N vs. | | | BPH vs. | BPH vs. | PIN vs. |
|---|---|---|---|---|---|---|
| Peak (Da) | BPH | N vs. PIN | N vs. PCA | PIN | PCA | PCA |
| 4036 | 0.317 | 0.083 | 0.059 | 0.025¹ | 0.039¹ | 0.102 |
| 4361 | 0.564 | 0.059 | 0.038¹ | 0.046¹ | 0.102 | 0.083 |
| 4413 | 0.083 | 0.157 | 0.317 | 0.655 | 1.000 | 0.317 |
| 4639 | 1.000 | 1.000 | 0.083 | 1.000 | 0.157 | 0.083 |
| 4749 | 0.317 | 0.034¹ | 0.102 | 0.059 | 0.109 | 0.564 |
| 5666 | 0.038¹ | 1.000 | 0.059 | 0.024² | 0.039² | 0.408 |
| 28422 | 0.083 | 0.041² | 0.066 | 0.680 | 0.180 | 0.564 |
| (PSA) | | | | | | |

¹significant p-values (≦0.05), based on over-expression as compared to N or BPH
²significant p-values (≦0.05), based on under-expression as compared to N or BPH

Since no single peptide or protein was discovered to be present, or differentially expressed in all the PIN or PCA profiles, various combinations of selected proteins were evaluated to identify a panel of markers (expression levels) that could improve disease classification of each specific cell type. A biomarker combination was classified as positive if any marker in the combination was present in a sample, and negative if none of the markers were detected in a specimen. Since these markers were over-expressed in both PCA and PIN, the combination of the markers did not improve the specificity for each diseased cell type individually. Better discrimination was achieved by combining the benign cell types (N, BPH) and comparing them to diseased cell types (PIN, PCA). The sensitivity and specificity of each marker for benign versus disease was then calculated, both individually and in additive combinations. As shown in Table 4, by combining markers EP2 (m/z 4036) and EP5 (m/z 4639), there was an improvement in the sensitivity for PCA from 71% to 86%, while maintaining a specificity of 100% for PIN or PCA combined. This combination of markers also identified 44% of the PIN samples. Combining marker EP3 (m/z 4361) and marker EP5 (m/z 4639) increased the sensitivity for PCA to 100%, while the specificity decreased to 87% for PIN and PCA. By combining markers EP3 and EP6 (m/z 4749), 100% of the PCA and 89% of the PIN samples could be correctly identified, with a slight decrease in specificity. Combinations involving three or more of the markers did not improve the overall specificity and sensitivity. The 5666 Da protein, (marker EP8, Table 4) had a sensitivity of 86% and specificity of 88% for detecting BPH. This marker also provided some discrimination between BPH and PIN lysates having 22% sensitivity and 68% specificity for PIN epithelia.

TABLE 4
Sensitivity and specificity of single markers and
combinations of markers for the detection of
abnormal (diseased) cells.

| | | Sensitivity | | Specificity | | Specificity |
|---|---|---|---|---|---|---|
| Marker | Expression* | PCA | PIN | PCA | PIN | PIN or PCA |
| EP2 | increase | 71% | 44% | 83% | 77% | 100% |
| | of m/z 4036 | | | | | |
| EP3 | increase | 71% | 44% | 75% | 68% | 87% |
| | of m/z | | | | | |
| | of 4361 | | | | | |
| EP5 | presence | 43% | 0% | 100% | — | 100% |
| | of m/z 4639 | | | | | |
| EP6 | increase | 57% | 67% | 71% | 71% | 93% |
| | of m/z | | | | | |
| | of 4749 | | | | | |
| Combi- | EP2 + EP3 | 86% | 67% | 67% | 64% | 87% |
| nation | | | | | | |
| | EP2 + EP5 | 86% | 44% | 83% | 77% | 100% |
| | EP3 + EP5 | 100% | 56% | 75% | 68% | 87% |
| | EP2 + EP6 | 86% | 78% | 67% | 68% | 93% |
| | EP3 + EP6 | 100% | 89% | 54% | 55% | 80% |
| | EP5 + EP6 | 86% | 67% | 71% | 68% | 93% |
| | EP2 + EP3 + | 86% | 67% | 67% | 64% | 93% |
| | EP5 | | | | | |
| | EP2 + EP3 + | 100% | 89% | 54% | 59% | 80% |
| | EP6 | | | | | |
| | | BPH | PIN | BPH | PIN | |
| EP8** | increase | 86% | 22% | 88% | 68% | |
| | of m/z 5666 | | | | | |

*The increase in abundance of each marker (EP2-EP6) found in PIN and PCA epithelial cells was determined from matched normal or BPH epithelial cell lysates.
**The sensitivity and specificity of a marker (EP8) found in abundance in BPH samples when compared to PIN and PCA samples.

Multivariable analysis of the peak expression data was evaluated to determine if the simultaneous over-expression or under-expression of several proteins in combination could be used to predict benign versus diseased cell lysates. Logistic regression analysis was performed with the seven most significant differentially expressed peaks (4036 Da, 4413 Da, 4639 Da, 4729 Da, 5666 Da, and 28422 (PSA)). Table 5 shows a predictive equation based on diseased (PIN/PCA.) versus benign (N/BPH), resulted in 93.3% specificity and 93.8% sensitivity for PIN or PCA cell lysates.

TABLE 5
Classification table based on logistic regression analysis using
expression levels of seven proteins 4036 Da (EP2), 4361
Da (EP3), 4413 Da (EP4), 4639 (EP5), 4749 Da
EP6), 5666 Da (EP8), 28422 Da (PSA).

predicted
PIN/PCA

| | | | |
|---|---|---|---|
| observed | N | Y | % correct |

| | | | | | |
|---|---|---|---|---|---|
| PIN/PCA | N | 15 | 14 | 1 | 93.3 (specificity) |
| | Y | 16 | 1 | 15 | 93.8 (sensitivity) |
| | | | | | 93.5 (overall) |

The development of cancer is a multistep process involving many events in the expression of oncogenes and tumor suppressor genes. Single changes in gene expression can lead to the differential expression of a myriad of proteins. Analyzing the multiple protein changes that occur in prostate disease progression is a difficult task, made even more daunting by the biological heterogeneity of the disease. Advances in time of flight mass spectroscopy, resulting in the SELDI technology, have lead to a method for the sensitive and direct analysis and profiling of proteins in complex biological samples. Previous studies in this and other laboratories have profiled a limited number of prostate cell specimens (see e.g. Paweletz, et al, Drug Development Research 49: 3442 (2001), Wright, et al, Prostate Cancer and Prostate Diseases 2: 264-267 (2000), and Paweletz, et al., Urology 57: 160-163 (2001)). In this study we combined SELDI with LCM to produce protein profiles from 40 prostate cell lysates derived from nine prostatectomy specimens. Our results demonstrate that the protein profiles of normal prostate epithelia, BPH, PIN and PCA display discriminating differences.

Protein extracts prepared from prostate cell types were analyzed using an IMAC3 protein biochip pretreated with CuSO₄. An average of 70 protein peaks were detected from the cell lysates using this surface. Overall there were a relatively large number of common peaks in the benign and diseased epithelial cell profiles, and very few peaks that would, based on presence or absence, be candidate biomarkers for the disease progression and/or diagnosis of prostate cancer. It was therefore determined that calculating abundance levels of peaks would enhance the significance of the results and identify possible expression differences between the samples. Expression levels were calculated for diseased cell profiles as compared to expression levels found in matched benign cell types. Out of the 70 peaks observed, 14 of these (20%) displayed deregulation in the diseased profiles.

Several small molecular weight peptides or proteins (3000 to 6000 Da) had increased expression levels in the PIN and PCA cell extracts. The cluster of peaks in this range could have originated from proteolysis, or may be cleavage products of larger proteins, but were consistent enough in common cell types to be considered part of the general profile. However, even cleavage products of proteins may be indicative of changes occurring in the prostate disease cycle, as demonstrated in a study of semenogelin, from seminal plasma (see Merchant, et al., Electrophoresis, 21: 1164-1177 (2000)). Furthermore, since the IMAC surface has the ability to bind phosphorylated peptides or proteins it is feasible that some of the changes observed are due to differential phosphorylation of the proteins or peptides in the diseased cell types. Two peaks identified in this study with an average mass 4827 Da±26.5 (EP7) (common in the benign cell types) and 4749 Da±26.1 (EP6) (overexpressed in PIN/PCA, see Table 1) may represent a de-phosphorylation event occurring in the PIN and PCA cell lysates. The average mass shift between these proteins is 78 Da, close to the calculated mass shift of 79 Da for a 5′ de-phosphorylation. Phosphorylation is one of the most important post-translational modifications of proteins and enables regulation of many physiological cell processes (transport, proliferation, differentiation). Examples of aberrant phosphorylation of proteins found in cancer studies include ERK ½ in breast cancer (see Gee, et al., Int. J Cancer 95: 247-254 (2001)), and androgen receptor in prostate cancer (see Wang, et al., Biochem. Biophys. Res. Commun., 259: 21-28 (1999).

Alternatively, these small molecules could themselves be intact functional proteins or peptides, examples of which include pro-hormones, growth factors, amidated peptides, and defensins. In a recent study by Rocchi et al., Cancer Res., 61: 1196-1206 (2001) PC-3 and Du145 prostate cancer cell lines were found to produce and secrete a multifunctional amidated peptide. Immunohistochemical staining was also performed on human prostate tissue and showed increased levels of adrenomedullin staining in cancerous epithelia. The authors further demonstrated the activity of the enzyme peptidylglycine alpha-amidatin monooxygenase (PAM) in prostate cancer cell lines, which produces alpha-amidated bioactive peptides from their inactive glycine-extended precursors. C-Terminal amidation is common in peptide hormones, and these peptides are produced in neuroendocrine cells, known to be present in prostate carcinoma, and function as growth factors.

Previous studies have shown a cytogenetic link between high grade PIN and Prostate Cancer strengthening its role as a precursor lesion (see Alcaraz, et al., Prostate, 47: 29-35 (2001)). In addition, greater than 50% of patients with high grade PIN have cancer detected on a subsequent biopsy (see Park, et al., J. Urol. 165: 1409-14140 (2001). Therefore, the identification of proteins associated with PIN may be useful as markers of early detection. In this study PIN and PCA cell lysates exhibited similar protein profiles. Three peaks at 4036 Da (EP2), 4361 Da (EP3), and 4749 Da (EP6), showed increased abundance in PIN and PCA lesions when compared to matched benign cell profiles. Two other peaks at 11744 (EP10), and 28442 Da (free PSA) had decreased abundance in BPH, PIN and PCA cell extracts, and one marker at 14696 Da (EP11) was over-expressed in 56% of the PIN samples and only 29% of matched PCA samples. Interestingly, a few of these peaks, based on closely matched molecular weights, were found to be present in a matched seminal plasma from one of the patients donating tissue for this study (data not shown). Since these seminal plasma profiles were generated using the IMAC surface pre-treated with CuSO₄, they may represent the same protein, however, additional matched seminal plasma samples will need to be examined to confirm this result. The identification of these peptides or proteins and their use as possible markers of early detection is currently under investigation in our laboratory.

The independent evolution of PIN and tumor foci is a common feature of primary tumors of the prostate, making a panel of biomarkers the most likely solution to improvements in early detection and diagnosis. A combination of biomarkers with the most significant differential expression was therefore investigated. Since most of the markers found in the PCA profiles were also present in the PIN profiles the ability to discriminate these two cell types from each other was difficult; whereas, better discrimination could be achieved between the benign (N, BPH) and the diseased cell types (PIN or PCA combined). However, a combination of markers 4361 Da (EP3), and 4749 Da (EP6) increased the sensitivity to 100% for detection of PIN and PCA with a only a slight decrease in specificity. Also of note, were two peaks (4639 Da (EP5)and 24184 Da (EP12)) found only in PCA cell lysates. Both of these markers were expressed in 43% of tumor samples. No correlation of biomarker expression with grade of tumor was observed. Further studies using different capture surfaces for SELDI profiling may reveal additional PCA biomarkers. Protein profiles of metastatic prostate samples will be required to determine if any of the selected “biomarkers” represent markers of metastatic potential.

SELDI analyses performed on cell lysates of surrounding adjacent normal epithelia as well as stroma was performed to determine if possible field effects accounted for the differences in the protein profiles. These analyses revealed interesting information on the milieu of proteins present in the diseased prostate cells. Defensins α-1 and α-2 were present in both epithelial and stroma cells with increased amounts in BPH, PIN and PCA cell lysates and surrounding stroma. Defensins are a family of small peptides with antimicrobial, cytotoxic, and antitumor activities (see Lichtenstein, et al., J. Immunol. 140: 2686-2694 (1988). Two types of defensins have been characterized. β-defensins are primarily expressed by epithelial cells of the kidneys, skin, and respiratory tract, (see Yang, et al., Science, 286: 525-528 (1999), and Zhao, et al., FEBS Lett., 396: 319-322 (1996)), whereas, α-defensins are expressed by neutrophils and Paneth cells (see Ayabe, et al., Nat. Immunol., 1: 113-118 (2000)). Recently a-defensins have been found in the submandibular glands of patients with oral carcinoma (see Mizukawa, et al., Anticancer Res., 20: 1125-1127, (2000), and Mizukawa, et al., Anticancer Res., 19: 2969-2971 (1999)), and, a study found α-defensin peptides in the urine of bladder cancer patients (see Vlahou, et al., Am. J. Pathol., 158: 1491-1502 (2001)). It is possible that neutrophils in the surrounding area are secreting these defensins into prostate tissue in response to an inflammatory or disease state. Unfortunately no tissue from a normal non-diseased prostate was available to use as a control to test this hypothesis. Another peak at 4361 Da (EP3), found to be over-expressed in the PIN and PCA cell extracts, was also present at higher levels in stroma adjacent to the diseased cells, suggesting a possible field effect for this peptide.

Differences were observed for the expression of a 28,400 Da peak, which is presumed to be intracellular free PSA The molecular weight identified in our study closely matches the observed molecular mass of 28,430 Da for free PSA determined using ion spray mass spectroscopy (ISMS) (see Belanger, et al., Prostate, 27: 187-197 (1995). This peak was also absent from matched stroma cell lysates, indicating its specific expression in epithelial cells. Furthermore, data obtained from an immunoassay, also performed using the SELDI platform, Wright, et al., Prostate Cancer and Prostate Diseases 2: 264-267 (2000), has identified this peak from LCM lysates as PSA based on immunoaffinity (data not shown). In this study, normal epithelia of the prostate expressed large amount of PSA whereas the diseased cell types (PIN and PCA.) had reduced expression levels. As expected, PSA was absent from stroma cells. Normal epithelia microdissected directly adjacent to the tumor foci also expressed high levels of PSA. This decrease in intracellular PSA in PCA cells is in agreement with other studies. For example, Stege et al., Prostate, 38: 183-188 (1999) found tissue PSA levels lower in cancerous than in normal tissue, and a study by Weir et al., J. Urol., 163: 1739-1742, (2000) found immunohistochemical staining intensity of PSA inversely correlated with histological grade of the tumor. Interestingly a recent report by Pawletz et al., Drug Development Research 49: 34-42 (2001), also found a 28 kDa protein peak (not identified as PSA) via SELDI to be down-regulated in microdissected PCA cells when compared to matched normal epithelia. Our result confirms the current opinion that the increase in serum PSA in men with prostate cancer is not due to increased production of PSA by the tumor cells, but an increased leakage or secretion of PSA into the circulation by the surrounding normal epithelium.

The BPH protein profiles also displayed some notable differences. There was an increase in defensin α-1 (3448 Da, EP1) in the BPH lesions, as well as an increase in abundance of peaks at 4413 Da (EP4), and 5666 Da (EP8). Of special interest was the over-expression of a peak at 5666 Da (EP8) found in greater abundance in 86% of the BPH cell lysates tested with a specificity of 88%. Only 22% of the PIN lesions, and none of the PCA lesions overexpressed this marker. Efforts are underway to characterize this marker. A protein biomarker indicative of BPH alone may, if secreted into serum or seminal plasma, be useful in the prevention of unnecessary biopsies in patients with elevated PSA. Two other high molecular weight proteins at 48308 Da (EP13) and 53830 Da (EP14) were also found at increased abundance in BPH cell lysates.

The present invention provides novel materials and methods for aiding prostate cancer diagnosis using markers that are differentially present in samples pure matched benign (normal or BPH), PIN (high grade), and/or PCA cells. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

What is claimed is:

1. A method of qualifying a prostate cancer status in a subject comprising:
(a) measuring at least one biomarker in a sample from the subject, wherein the biomarker is selected from the group consisting of:
Marker EP1: 3448±19 Da,
Marker EP2: 4036±22 Da,
Marker EP3: 4361±24 Da,
Marker EP4: 4413±24 Da,
Marker EP5: 4639±26 Da,
Marker EP6: 4749±26 Da,
Marker EP7: 4827±27 Da,
Marker EP8: 5666±31 Da,
Marker EP9: 8445±46 Da,
Marker EP10: 11744±65 Da,
Marker EP11: 14696±81 Da,
Marker EP12: 24184±133 Da,
Marker EP13: 48308±266 Da,
Marker EP14: 53830±296 Da; and combinations thereof; and,
(b) correlating the measurement with prostate cancer status.

2. The method of claim 1, wherein the prostate cancer status is selected from the group consisting of prostate cancer (PCA.), prostate intraepithelial neoplasia (PIN), and benign prostate hyperplasia (BPH).

3. The method of claim 1, wherein the sample is prostate tissue extract.

4. The method of claim 1, wherein measuring comprises determining the mass of the protein.

5. The method of claim 4, wherein the mass of the protein is determined by mass spectrometry.

6. The method of claim 1, wherein the sample is selected from the group consisting of blood, serum, urine, prostatic fluid, seminal fluid, semen, and prostate tissue.

7. The method of claim 5, wherein mass spectrometry is gas phase ion spectrometry.

8. The method of claim 7, wherein gas phase ion spectrometry is laser desorption ionization mass spectrometry.

9. The method of claim 1, wherein step a) further comprises detecting the marker by immunoassay.

10. The method of claim 1, wherein
step a) further comprises:
i) fractionating the sample;
ii) binding a fraction of the sample to an adsorbent; and,
iii) comprises detecting the marker by gas phase ion spectrometry.

11. The method of claim 10, wherein the adsorbent is selected from the group consisting of a hydrophilic adsorbent, a metal chelate adsorbent, and a strong anion exchange adsorbent.

12. The method of claim 1, wherein
step a) comprises:
i) embedding a portion of a tissue specimen harvested from a patient in OCT and freezing the specimen;
ii) obtaining cryosections from the tissue specimen;
iii) obtaining cell samples from the cryosections by laser capture microdissection;
iv) mixing cell samples from step (d) with lysis buffer thereby producing cell lysates;
v) diluting and vortexing the cell lysates,
vi) centrifuging the vortexed cell lysates thereby producing a supernatant fraction;
vii) binding the supernatant fraction to an adsorbent; and,
viii) comprising detecting the marker using gas phase ion spectrometry.

13. The method of claim 12, wherein the adsorbent of step a) is selected from the group consisting of a hydrophilic adsorbent, a metal chelate adsorbent, and a strong anion exchange adsorbent.

14. The method of claim 1, wherein the sample comprises the marker EP8.

15. The method of claim 1, wherein the sample comprises the markers EP2 and EP3.

16. The method of claim 1, wherein the sample comprises the markers EP2 and EP5.

17. The method of claim 1, wherein the sample comprises the markers EP3 and EP5.

18. The method of claim 1, wherein the sample comprises the markers EP2 and EP6.

19. The method of claim 1, wherein the sample comprises the markers EP3 and EP6.

20. The method of claim 1, wherein the sample comprises the markers EP5 and EP6.

21. The method of claim 1, wherein the sample comprises the markers EP2,EP3 and EP5.

22. The method of claim 1, wherein the sample comprises the markers EP2,EP3 and EP6.

23. The method of claim 1, wherein the sample comprises the markers EP2, EP3, EP4, EP5, EP6, and EP8.

24. The method of claim 8, wherein
step b) comprises:
i) generating data for each marker with the mass spectrometer, the data comprising a mass/charge ratio and an amount determination for each ion corresponding to each marker;
ii) transforming the data into computer-readable form; and,
iii) executing an algorithm with a programmable digital computer,
wherein the algorithm determines closeness-of-fit between the computer-readable data and a data set indicating a diagnosis of PCA, PIN, BPH or a negative diagnosis.

25. A method for detecting at least one marker in a sample, the method comprising:
a) obtaining a sample comprising at least one marker, where each marker is selected from the group consisting of:
Marker EP1: 3448±19 Da,
Marker EP2: 4036±22 Da,
Marker EP3: 4361±24 Da,
Marker EP4: 4413±24 Da,
Marker EP5: 4639±26 Da,
Marker EP6: 4749±26 Da,
Marker EP7: 4827±27 Da,
Marker EP8: 5666±31 Da,
Marker EP9: 8445±46 Da,
Marker EP10: 11744±65 Da,
Marker EP11: 14696±81 Da,
Marker EP12: 24184±133 Da,
Marker EP13: 48308±266 Da, and
Marker EP14: 53830±296 Da; and,
b) detecting the marker by gas phase ion spectrometry.

26. The method of claim 25, wherein gas phase ion spectrometry is laser desorption/ionization mass spectrometry.

27. The method of claim 22, wherein the sample comprises at least two markers wherein each marker is differentially present in the sample.

28. The method of claim 26, further comprising:
c) generating data for each marker with the mass spectrometer, the data comprising a mass/charge ratio and an amount determination for each ion corresponding to each marker,
d) transforming the data into computer-readable form; and
e) executing an algorithm with a programmable digital computer
wherein the algorithm detects the amount determination in the computer-readable data representing the marker and determines closeness-of-fit between the computer-readable data and a data set indicating a diagnosis of PCA, PIN, BPH or a negative diagnosis.

29. The method of claim 29, wherein the algorithm comprises an artificial intelligence program.

30. The method of claim 29, wherein the artificial intelligence program is a fuzzy logic, cluster analysis or neural network.

31. The method of claim 25, wherein step a) further comprises: fractionating the sample by size exclusion chromatography or anion exchange chromatography, and collecting a fraction that includes the marker or markers.

32. The method of claim 25, wherein step a) further comprises contacting the sample with a substrate comprising an adsorbent that retains the marker and removing unretained sample.

33. The method of claim 29, wherein the substrate is a mass spectrometer probe comprising the adsorbent on a probe surface.

34. The method of claim 29, wherein the substrate is a resin, and step a) further comprises placing the resin with the marker retained by the adsorbent on a mass spectrometer probe.

35. The method of claim 29, wherein the adsorbent is selected from the group consisting of a hydrophilic adsorbent, a strong anion exchange adsorbent and a metal chelate adsorbent.

36. The method of claim 26, wherein
step a) further comprises:
i) providing a probe adapted for use with a mass spectrometer comprising an adsorbent attached thereto;
ii) contacting the sample comprising the marker with the adsorbent.

37. The method of claim 26, wherein
step a) further comprises:
i) providing a substrate comprising an adsorbent attached thereto;
ii) contacting the sample comprising the protein with the adsorbent;
iii) placing the substrate on a probe adapted for use with a mass spectrometer.

38. The method of claim 36, wherein the adsorbent is a hydrophilic adsorbent or a metal chelate adsorbent.

39. The method of claim 37, wherein the adsorbent is a hydrophilic adsorbent comprising silicon oxide.

40. The method of claim 37, wherein the adsorbent is a metal chelate adsorbent comprising copper.

41. The method of claim 37, wherein the adsorbent comprises an antibody that specifically binds to the marker.

42. A purified marker selected from the group consisting of:
Marker EP1: 3448±19Da,
Marker EP2: 4036±22 Da,
Marker EP3: 4361±24 Da,
Marker EP4: 4413±24 Da,
Marker EP5: 4639±26 Da,
Marker EP6: 4749±26 Da,
Marker EP7: 4827±27 Da,
Marker EP8: 5666±31 Da,
Marker EP9: 8445±46 Da,
Marker EP10: 11744±65 Da,
Marker EP11: 14696±81 Da,
Marker EP12: 24184±133 Da,
Marker EP13: 48308±266 Da, and
Marker EP14: 53830±296 Da.

43. The purified protein of claim 42, produced by a process comprising:
i) fractionating a sample comprising the marker or markers by size exclusion chromatography or anion exchange chromatography, and,
ii) collecting a fraction that includes the marker or markers.

44. The purified protein of claim 42, produced by a process comprising:
i) microdissecting a cell sample comprising the marker by laser capture microdissection, thereby producing isolated cells,
ii) lysing the isolated cells producing a cell lysate,
iii) centrifuging the cell lysate, thereby producing a cell supernatant comprising the marker,
iv) contacting the cell supernatant to an adsorbent sufficient to allow the adsorbent to bind the marker;
v) washing the adsorbent to remove unbound cell supernatant; and,
vi) eluting the marker from the adsorbent.

45. A kit comprising:
(1) an adsorbent attached to a substrate, wherein the adsorbent is suitable for retaining a marker selected from the group consisting of:
Marker EP1: 3448±19 Da,
Marker EP2: 4036±22 Da,
Marker EP3: 4361±24 Da,
Marker EP4: 4413±24 Da,
Marker EP5: 4639±26 Da,
Marker EP6: 4749±26 Da,
Marker EP7: 4827±27 Da,
Marker EP8: 5666±31 Da,
Marker EP9: 8445±46 Da,
Marker EP10: 11744±65 Da,
Marker EP11: 14696±81 Da,
Marker EP12: 24184±133 Da,
Marker EP13: 48308±266 Da, and
Marker EP14: 53830±296 Da; and
(2) instructions for using the substrate to detect the marker.

46. The kit of claim 45, wherein the instructions include methods for contacting a sample comprising the marker with the adsorbent.
